(19) United States
(12) Patent Application Publication
(10) Pub. No.: US 2008/0255052 A1
(43) Pub. Date: Oct. 16, 2008

(54) Immunologic regulation by theta defensins

(75) Inventors: Michael E. Selsted, Irvine, CA (US); Dat Q. Tran, Anaheim, CA (US)

Correspondence Address: MCDERMOTT, WILL & EMERY, 4370 LA JOLLA VILLAGE DRIVE, SUITE 700, SAN DIEGO, CA, 92122, US

(73) Assignee: The Regents of the University of California, Oakland, CA (US)

(21) Appl. No.: 12/006,991

(22) Filed: Jan. 7, 2008

Related U.S. Application Data
(63) Continuation of application No. 11/316,316, filed on Dec. 21, 2005, now abandoned.
(60) Provisional application No. 60/638,728, filed on Dec. 23, 2004.

(51) Int. Cl. A61K 38/00; A61P 43/00

(52) U.S. Cl. 514/13; 514/14; 514/15

(57) ABSTRACT

The invention provides a method of inhibiting undesirable microbial contamination, invasion, or growth in an individual by administering to the individual an effective amount of a theta defensin. The invention additionally provides a method of inhibiting deleterious effects resulting from microbial contamination, invasion, or growth in an individual by administering to an individual an effective amount of a theta defensin, whereby immune-mediated pathology is limited and immune function is improved.

This application claims the benefit of priority of U.S. Provisional application Ser. No. 60/638,728, filed Dec. 23, 2004, the entire contents of which is incorporated herein by reference.

This invention was made with government support under Grant No. AI22931, awarded by the National Institutes of Health. The government has certain rights in the invention.

### BACKGROUND OF THE INVENTION

The present invention relates generally to antimicrobial agents and, more specifically, to cyclic theta defensin peptides and methods of using a theta defensin peptide to reduce or inhibit microbial growth or survival and to limit the pathologic sequalae of microbial infection.

Infections by microorganisms, including bacteria, viruses and fungi, are a major cause of human morbidity and mortality. Although anyone can be a victim of such infection, the sick and elderly are particularly susceptible. For example, hospitalized patients frequently acquire secondary infections due to a combination of their weakened condition and the prevalence of microorganisms in a hospital setting. Such opportunistic infections result in increased suffering of the patient, increased length of hospitalization and, consequently, increased costs to the patient and the health care system. Similarly, the elderly, particularly those living in nursing homes or retirement communities, are susceptible to infections because of their close living arrangement and the impaired responsiveness of their immune systems.

Numerous drugs are available for treating infections by certain microorganisms. In particular, various bacterial infections have been amenable to treatment by antibiotics. However, the prolonged use of antibiotics since their discovery has resulted in the selection of bacteria that are relatively resistant to these drugs. Furthermore, few if any drugs are effective against microorganisms such as viruses. As a result, continuing efforts are being made to identify new and effective agents for treating infections by a variety of microorganisms.

The identification of naturally occurring compounds that act as antimicrobial agents has provided novel and effective drugs. Many organisms protect themselves by producing natural products that are toxic to other organisms. Frogs, for example, produce a class of peptides, magainins, which provide a defense mechanism for the frog against potential predators. Magainins have been purified and shown to have antimicrobial activity, thus providing a natural product useful for reducing or inhibiting microbial infections.

Natural products useful as antimicrobial agents also have been purified from mammalian organisms, including humans. For example, the defensins are a class of peptides that have been purified from mammalian neutrophils and demonstrated to have antimicrobial activity. Similarly, indolicidin is a peptide that has been isolated from bovine neutrophils and has antimicrobial activity, including activity against viruses, bacteria, fungi and protozoan parasites. Thus, naturally occurring compounds provide a source of drugs that are potentially useful for treating microbial infections.

Upon identifying naturally occurring peptides useful as antimicrobial agents, efforts began to chemically modify the peptides to obtain analogs having improved properties. Such efforts have resulted, for example, in the identification of indolicidin analogs which, when administered to an individual, have increased selectivity against the infecting microorganisms as compared to the individual's own cells. Thus, the availability of naturally occurring antimicrobial agents has provided new drugs for treating microbial infections and has provided a starting material to identify analogs of the naturally occurring molecule that have desirable characteristics.

Sepsis is a systemic inflammatory response syndrome (SIRS) triggered by an infection (Rice and Bernard, Ann. Rev. Med. 56:225-248 (2005) (published online August 2004 in Review in Advance; Hotchkiss and Karl, NEJM 348:138-150 (2003); Wang et al., J. Int. Med. 255:320-331 (2004)). Sepsis syndrome afflicts almost 750,000 patients in the United States each year, costing almost $17 billion and causing 210,000 deaths annually. The high mortality rate has become even more problematic as the number of antibiotic resistant bacteria has grown. Patients who develop severe sepsis have mortality rates as high as 50%.

Although natural products and their analogs have provided new agents for treating microbial infections, it is well known that microorganisms can become resistant to drugs. Thus, a need exists to identify agents that effectively reduce or inhibit the growth or survival of microorganisms and that further can be used to treat microbial infections, including sepsis. The present invention satisfies this need and provides additional advantages.

### SUMMARY OF THE INVENTION

The invention provides a method of inhibiting undesirable microbial colonization, invasion, or/or growth in an individual by administering to the individual an effective amount of a theta defensin, whereby microbial growth is inhibited. The invention additionally provides a method of inhibiting a deleterious effect of microbial growth in an individual by administering to an individual an effective amount of a theta defensin, whereby microbial growth is inhibited and/or the pathologic sequalae resulting from the host response to the organism is mitigated.

### BRIEF DESCRIPTION OF THE DRAWINGS

FIG. 1 shows the amino acid sequences of theta-defensins, PG-1, and analogs thereof (see U.S. publication 20040014669). Native sequences are indicated by asterisks (*). The peptide charges are calculated at pH 7.0. Cysteine (shaded residues) connectivity is shown in accordance to structures determined for PG-1 and RTD-1 (Kokryakov et al., FEBS Letters 327:231-236 (1993); Tang et al., Science 286:498-502 (1999)). “a”-acyclic analogs of theta-defensins; “c”-cyclic analogs of PG-1. “-NH” or “-OH” denotes amide and acid of the peptide terminus, respectively. “3cys”-tridisulfide PG-1 analogs. “Even” and “overlapping” chain termini are indicated by a pair of numbers separated by a colon corresponding to respective lengths of N- and C-termini. Native PG-1 and PG-1-OH have the 5:3, “overlapping”-termini structure. The designation of all RTD-2 peptides (peptide ID's #4, #7, #8, #9, #10 in FIG. 1) and RTD-3 peptides (peptide ID's #5 and #6) were revised from previous U.S. Patent publication 20040014669 to match the nomenclature in published references (Leonova et al., L., V. Kokryakov, et al. J. Leukocyte Biol. 70: 461-464 (2001); Tran et al. J. Biol. Chem. 277:3079-3084 (2002)).

FIG. 2 shows the microbicidal activity of theta defensins against Escherichia coli.

FIG. 3 shows the microbicidal activity of theta defensins against Staphylococcus aureus.

FIG. 4 shows the microbicidal activity of theta defensins against Candida albicans.

FIG. 5 shows the antimicrobial activities of various theta defensins against E. coli measured using an antimicrobial diffusion assay.

FIG. 6 shows the antimicrobial activities of various theta defensins against S. aureus measured using an antimicrobial diffusion assay.

FIG. 7 shows the antimicrobial activities of various theta defensins against C. albicans measured using an antimicrobial diffusion assay.

FIG. 8 shows the antimicrobial activities of various theta defensins against Cryptococcus neoformans measured using an an antimicrobial diffusion assay.

FIGS. 9A-9C show staphylocidal activities of theta defensins and protegrin (PG-1) in various buffers and inclusion of physiologic sodium chloride.

FIGS. 10A-10C show bactericidal activities of theta defensins and PG-1 in the presence of physiologic CaCl₂ and MgCl₂.

FIGS. 11A and 11B show bactericidal activities of theta defensins and PG-1 in the presence of serum.

FIG. 12 shows the structures of RTD-1 and an acyclic version, aRTD-1-Hse, which were tested for anti-HIV-1 activity.

FIG. 13 shows the anti-HIV activity of theta and beta defensins.

FIG. 14 shows the inhibitory effect of theta defensins on β-galactosidase activity.

FIG. 15 shows a cytotoxicity assay of various theta defensins against HS68 fibroblasts.

FIG. 16 shows a cytotoxicity assay of various theta defensins and protegrin PG-1 OH against L929 and Hela 929 cells.

FIG. 17 shows a red blood cell hemolysis assay of various theta defensins.

FIGS. 18A and 18B show survival curves of mice subjected to cecal ligation and puncture that are treated with various theta defensins in a model of polymicrobial peritonitis.

FIG. 19 shows the modulatory effect of theta defensin RTD-1 on tumor necrosis alpha (TNFα) release from peripheral blood mononuclear cells (PBMCs).

FIG. 20 shows transduction of an HIV TAT peptide-cyrptdin 4 fusion (TAT-Crp-4) and uptake in 293 cells.

### DETAILED DESCRIPTION OF THE INVENTION

The invention provides the use of theta defensins and theta defensin analogs having antimicrobial activity for inhibiting microbial growth and, for example, treating an individual having an infection. The theta defensins and theta defensin analogs can be used to reduce or inhibit the growth or survival of a microorganism. In addition to antimicrobial activity, the theta defensins and theta defensin analogs exhibit low hemolytic activity. Furthermore, the theta defensins are particularly effective at treating infections in an individual, including sepsis.

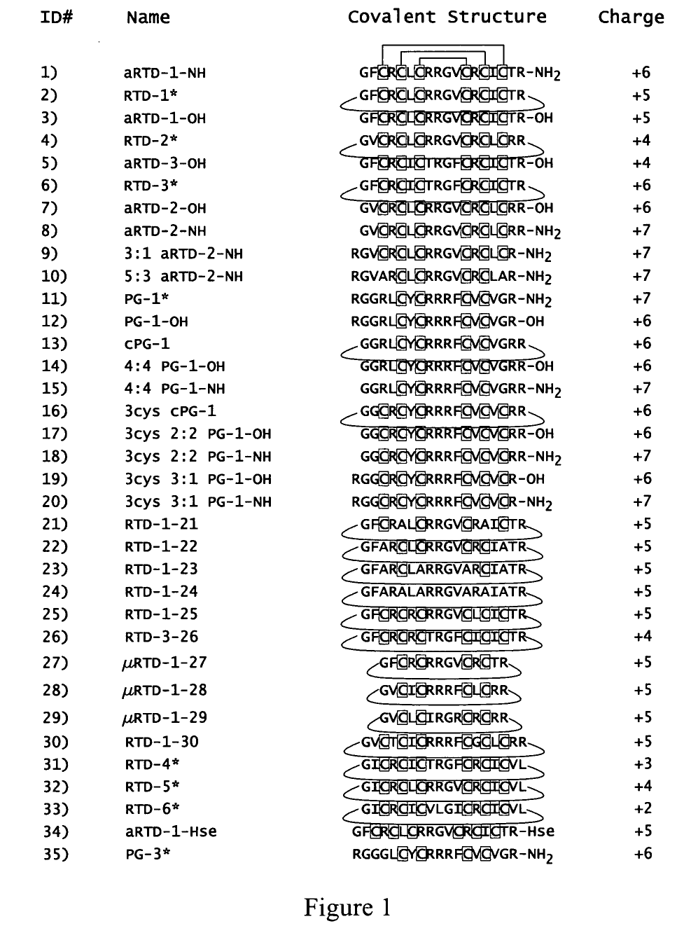

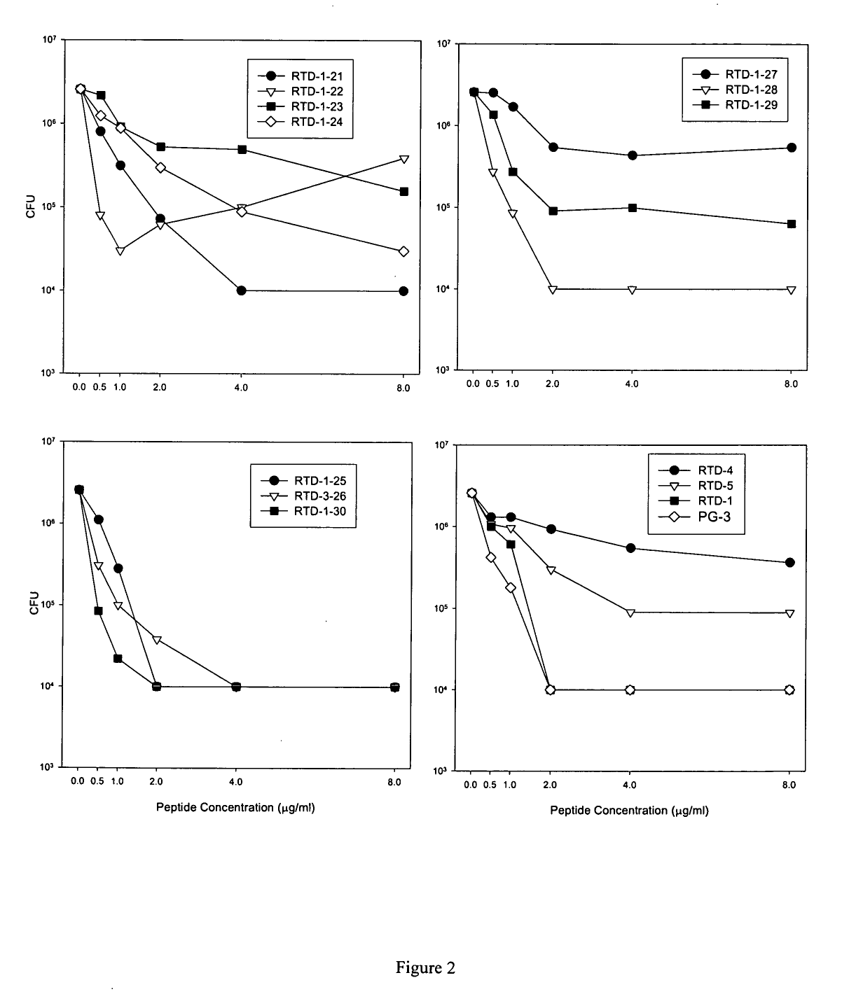

The Rhesus theta defensin RTD-1 is a macrocyclic 18-amino acid antimicrobial peptide formed by the ligation of two nine-residue sequences derived from similar 76-amino acid precursors, termed RTD1a and RTD1b (see FIG. 2) (U.S. Pat. No. 6,335,318, issued Jan. 1, 2002; U.S. Pat. No. 6,514,727, issued Feb. 4, 2003; WO 00/68265; Tang et al., Science 286:498-502 (1999); U.S. publications 20040014669 and 20030162718, each of which is incorporated herein by reference). The two nine-residue sequences can be ligated as a heterodimer (RTD-1, RTD-4, and RTD-5) or homodimer (RTD-2, RTD-3, and RTD-6) (see FIG. 1). Naturally occurring theta defensins include RTD-1, RTD-2 and RTD-3, RTD-4, RTD-5, and RTD-6 (see U.S. publication 20040014669). As used herein, the term “theta defensin” includes naturally occurring theta defensins as well as theta defensin analogs as described, for example, in U.S. publication 20040014669.

The theta defensin peptides of the invention have antimicrobial activity and include theta defensin and theta defensin analogs having the amino acid sequence Xaa1-Xaa2-Xaa3-Xaa4-Xaa5-Xaa1-Xaa6-Xaa4-Xaa4-Xaa1-Xaa1-Xaa6-Xaa4-Xaa5-Xaa1-Xaa3-Xaa7-Xaa8; having the amino acid sequence Xaa1-Xaa2-Xaa3-Xaa4-Xaa5-Xaa1-Xaa6-Xaa7-Xaa8-Xaa1-Xaa2-Xaa6-Xaa4-Xaa5-Xaa1-Xaa3-Xaa7-Xaa8; and having the amino acid sequence Xaa1-Xaa1-Xaa3-Xaa4-Xaa5-Xaa1-Xaa6-Xaa4-Xaa4-Xaa1-Xaa1-Xaa6-Xaa4-Xaa5-Xaa1-Xaa3-Xaa4-Xaa4, wherein Xaa1 independently is an aliphatic amino acid; Xaa2 is an aromatic amino acid; Xaa3 is Cys or Trp; Xaa4 independently is Arg or Lys; Xaa5 is Cys or Trp; Xaa6 is Cys or Trp; Xaa7 is Thr or Ser; and Xaa8 is Arg or Lys. For example, Xaa1 can be an aliphatic amino acid such as Gly, Ile, Leu, Val or Ala and Xaa2 can be an aromatic amino acid such as Phe, Trp or Tyr. In general, a theta defensin is a cyclic peptide, wherein Xaa1 is linked through a peptide bond to Xaa8, and contains three intrachain crosslinks, which are formed between Xaa3 and Xaa3, between Xaa5 and Xaa5, and between Xaa7 and Xaa7. However, as disclosed herein, the invention also encompasses linear or acyclic theta defensins, including theta defensin precursors, as well as peptide portions of a theta defensin or theta defensin analog.

The theta defensin peptides of the invention have antimicrobial activity and include theta defensin and theta defensin analogs having the amino acid sequence Xaa1-Xaa1-Xaa3-Xaa4-Xaa5-Xaa1-Xaa3-Xaa7-Xaa8-Xaa1-Xaa2-Xaa3-Xaa4-Xaa3-Xaa1-Xaa3-Xaa1-Xaa1; and having the amino acid sequence Xaa1-Xaa1-Xaa3-Xaa4-Xaa5-Xaa1-Xaa6-Xaa4-Xaa4-Xaa1-Xaa1-Xaa6-Xaa4-Xaa3-Xaa1-Xaa3-Xaa1-Xaa1; and having the amino acid sequence Xaa1-Xaa1-Xaa3-Xaa4-Xaa3-Xaa1-Xaa3-Xaa1-Xaa1-Xaa1-Xaa1-Xaa3-Xaa4-Xaa3-Xaa1-Xaa3-Xaa1-Xaa1 wherein Xaa1 independently is an aliphatic amino acid; Xaa2 is an aromatic amino acid; Xaa3 is Cys or Trp; Xaa4 independently is Arg or Lys; Xaa5 is Cys or Trp; Xaa6 is Cys or Trp; Xaa7 is Thr or Ser; and Xaa8 is Arg or Lys. For example, Xaa1 can be an aliphatic amino acid such as Gly, Ile, Leu, Val or Ala and Xaa2 can be an aromatic amino acid such as Phe, Trp or Tyr. In general, a theta defensin is a cyclic peptide, wherein Xaa1 is linked through a peptide bond to Xaa8, and contains three intrachain crosslinks, which are formed between Xaa3 and Xaa3, between Xaa5 and Xaa5, and between Xaa7 and Xaa7.

As used herein, the term “independently,” when used in reference to the selection of an amino acid at a position in the generic structure of a theta defensin, means that the selection of one amino acid at a position, for example, Xaa1 at position 1 of the theta defensin sequence Xaa1-Xaa2-Xaa3-Xaa4-Xaa5-Xaa1-Xaa6-Xaa4-Xaa4-Xaa1-Xaa1-Xaa6-Xaa4-Xaa5-Xaa1-Xaa3-Xaa7-Xaa8; or of the theta defensin sequence Xaa1-Xaa2-Xaa3-Xaa4-Xaa5-Xaa1-Xaa6-Xaa7-Xaa8-Xaa1-Xaa2-Xaa6-Xaa4-Xaa5-Xaa1-Xaa3-Xaa7-Xaa8; or of the theta defensin sequence Xaa1-Xaa1-Xaa3-Xaa4-Xaa5-Xaa1-Xaa6-Xaa4-Xaa4-Xaa1-Xaa1-Xaa6-Xaa4-Xaa5-Xaa1-Xaa3-Xaa4-Xaa4, has no influence on the selection, for example, of Xaa1 at position 6 or 10 or the like. For example, Xaa1 can be Gly at position 1 and can be Leu or Ile at position 6.

The theta defensins and theta defensin analogs of the invention exhibit broad spectrum antimicrobial activity. An exemplified theta defensin is an 18 amino acid cyclic peptide having the amino acid sequence Gly-Phe-Cys-Arg-Cys-Leu-Cys-Arg-Arg-Gly-Val-Cys-Arg-Cys-Ile-Cys-Thr-Arg (SEQ ID NO:2), wherein the Gly at position 1 (Gly-1) is linked through a peptide bond to Arg-18, and wherein three intrachain crosslinks are present due to disulfide bonds between Cys-3 and Cys-16, between Cys-5 and Cys-14, and between Cys-7 and Cys-12. Other exemplary theta defensins include RTD-2 (SEQ ID NO:4) and RTD-3 (SEQ ID NO:6) (see FIG. 1).

A theta defensin which lacks free amino and carboxyl termini is resistant to exopeptidases and is thus relatively stable to proteolytic degradation. The invention also provides theta defensin analogs having antimicrobial activity (see Examples). Exemplary theta defensin analogs include the analogs shown in FIG. 1.

As used herein, the term “isolated,” when used in reference to a natural theta defensin, means that the peptide is free of at least a portion of the contents associated with or occurring with the theta defensin peptide in the native environment. An isolated theta defensin can be relatively free of proteins, lipids, nucleic acids or other molecules it normally is associated with in a cell. In general, an isolated theta defensin peptide can constitute at least about 25% by weight of a sample containing the theta defensin, and usually constitutes at least about 50%, at least about 75%, at least about 85%, at least about 90% of a sample, particularly about 95% of the sample or 99% or more. An isolated theta defensin can be obtained by isolation from a cell expressing the theta defensin, can be chemically synthesized, or can be expressed from a recombinant nucleic acid molecule (see U.S. Pat. Nos. 6,335,318 and 6,514,727; WO 00/68265; U.S. publications 20040014669 and 20030162718). Following chemical synthesis or recombinant expression, the theta defensin precursor peptide generally is linear and, therefore, can be further subjected to appropriate conditions for cyclizing the peptide and forming the intrachain crosslinks, as described previously and herein.

The theta defensin peptides shown as SEQ ID NOS:2, 4, 6, 31, 32, and 33, constitute the first members of a new class of defensins and are the basis for constructing theta defensin analogs as disclosed herein (see U.S. Pat. Nos. 6,335,318 and 6,514,727; WO 00/68265; U.S. publications 20040014669 and 20030162718). Previously described defensins are cationic, arginine-rich peptides having 29 to 42 amino acids and containing three disulfide bonds (see Lehrer et al., Cell 64:229-230 (1991); Lehrer and Ganz, Current Opin. Immunol. 11:23-27 (1999)). The β-defensins, for example, contain 38 to 42 amino acids and have a net charge of +4 to +10 (see U.S. Pat. No. 5,459,235, issued Oct. 17, 1995, which is incorporated herein by reference). The disulfide bonds in β-defensins are formed in a characteristic pattern between the first and fifth Cys residues, the second and fourth Cys residues, and the third and sixth Cys residues. In addition, some β-defensins contain a pyroglutamate residue at the amino terminus (U.S. Pat. No. 5,459,235, supra, 1995).

Defensins and defensin-like peptides are endogenously expressed in various organisms. In mammals, defensins generally are expressed in neutrophils, macrophages and intestinal cells, and other epithelia (see Lehrer et al., supra, 1991; Lehrer and Ganz, supra, 1999). Defensins can exhibit potent antimicrobial activity against a broad spectrum of microorganisms, including gram negative and gram positive bacteria, fungi, protozoans such as Acanthamoeba and Giardia, enveloped viruses such as herpes simplex viruses and human immunodeficiency viruses, and helminths. Defensins also have other properties, including chemotactic activity for human monocytes and the ability to interfere with adrenocorticotropin binding to its receptor (see Lehrer et al., supra, 1991).

Examples of viruses that have been found to cause infections in humans include but are not limited to: Retroviridae (for example, human immunodeficiency viruses, such as HIV-1 (also referred to as HTLV-I11), HIV-2, LA V or IDLV-III/LA V, or HIV-ill, and other isolates, such as HIV-LP); Picornaviridae (for example, polio viruses, hepatitis A virus; enteroviruses, human Coxsackie viruses, rhinoviruses, echoviruses); Calciviridae (for example, strains that cause gastroenteritis); Togaviridae (for example, equine encephalitis viruses, rubella viruses); Flaviviridae (for example, dengue viruses, encephalitis viruses, yellow fever viruses); Coronaviridae (for example, coronaviruses); Rhabdoviridae (for example, vesicular stomatitis viruses, rabies viruses); Filoviridae (for example, ebola viruses); Paramyxoviridae (for example, parainfluenza viruses, mumps virus, measles virus, respiratory syncytial virus); Orthomyxoviridae (for example, influenza viruses); Bungaviridae (for example, Hantaan viruses, bunga viruses, phleboviruses and Nairo viruses); Arena viridae (hemorrhagic fever viruses); Reoviridae (for example, reoviruses, orbiviurses and rotaviruses); Bimaviridae, Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus); Poxyiridae (variola viruses, vaccinia viruses, pox viruses); and Iridoviridae (for example, African swine fever virus); and unclassified viruses (for example, the etiological agents of Spongiform encephalopathies, the agent of delta hepatitis (thought to be a defective satellite of hepatitis B virus), the agents of non-A, non-B hepatitis (class 1=enterally transmitted; class 2=parenterally transmitted (i.e., Hepatitis C); Norwalk and related viruses, and astroviruses).

A new class of defensins, termed theta defensins, have been described (U.S. Pat. No. 6,335,318, issued Jan. 1, 2002; WO 00/68265; Tang et al., Science 286:498-502 (1999)). Theta defensins have been classified as members of the defensin family of peptides based on their cationicity, arginine-rich composition and the presence of three intrapeptide disulfide bonds, as well as their broad spectrum antimicrobial activity. However, theta defensins are distinguishable from previously described defensins in that naturally occurring theta defensins are cyclic peptides, which lack a free amino or carboxyl terminus, and are shorter than previously described defensins. Theta defensins have been described previously (see, for example, Owen et al. AIDS Res Hum Retroviruses 20:1157-1165 (2004); Yang et al., Infect. Genet. Evol. 5:11-15 (2005); Cole et al., Curr. Protein Pept. Sci. 5:373-381 (2004); Selsted, Curr. Protein Pept. Sci. 5:365-371 (2004); Wang et al., J. Immunol. 173:515-520 (2004); Owen et al., J. Pept. Res. 63:469-476 (2004); Abuja et al., FEBS Lett. 566:301-306 (2004); Yasin et al., J. Virol. 78:5147-5156 (2004); Nguyen et al., Peptides 24:1647-1654 (2003); Munk et al., AIDS Res. Hum. Retroviruses 19:875-881 (2003); Wang et al., J. Immunol. 170:4708-4716 (2003); Cole et al., Proc. Natl. Acad. Sci. USA 99:1813-1818 (2002); Tran et al., J. Biol. Chem. 277:3079-3084 (2002); Leonova et al., J. Leukoc. Biol. 70:461-464 (2001); Trabi et al., Biochemistry 40:4211-4221 (2001); Tam et al., Biochem. Biophys. Res. Commun. 267:783-790 (2000); Tang et al., Science 286:498-502 (1999)).

The theta defensins are exemplified by the peptides shown in FIG. 1. RTD-1 contains 18 amino acids, wherein the amino terminus of the first amino acid (Gly) is linked to the carboxyl terminus of the last amino acid (Arg) through a peptide bond, and wherein disulfide bonds are formed between Cys-3 and Cys-16, Cys-5 and Cys-14, and Cys-7 and Cys-12. For convenience of discussion, reference to an amino acid position in a theta defensin, or an analog thereof, is made with respect to the amino acid position in the linear form of theta defensin shown as FIG. 1 or of the theta defensin sequence Xaa1-Xaa2-Xaa3-Xaa4-Xaa5-Xaa1-Xaa6-Xaa4-Xaa4-Xaa1-Xaa1-Xaa6-Xaa4-Xaa5-Xaa1-Xaa3-Xaa7-Xaa8; or of the theta defensin sequence Xaa1-Xaa2-Xaa3-Xaa4-Xaa5-Xaa1-Xaa6-Xaa7-Xaa8-Xaa1-Xaa2-Xaa6-Xaa4-Xaa5-Xaa1-Xaa3-Xaa7-Xaa8; or of the theta defensin sequence Xaa1-Xaa1-Xaa3-Xaa4-Xaa5-Xaa1-Xaa6-Xaa4-Xaa4-Xaa1-Xaa1-Xaa6-Xaa4-Xaa5-Xaa1-Xaa3-Xaa4-Xaa4. As such, the amino acids are referred to as positions 1 through 18, starting with the Gly residue in (for example, position 1 of RTD-1 in FIG. 1) and ending with Arg (position 18).

A theta defensin can be obtained by purification of the native peptide from a natural source by expression of a recombinant theta defensin, or by chemical synthesis (see U.S. Pat. Nos. 6,335,318 and 6,514,727; WO 00/68265; U.S. publications 20040014669 and 20030162718). A theta defensin as shown in FIG. 1, or of the theta defensin sequence Xaa1-Xaa2-Xaa3-Xaa4-Xaa5-Xaa1-Xaa6-Xaa4-Xaa4-Xaa1-Xaa1-Xaa6-Xaa4-Xaa5-Xaa1-Xaa3-Xaa7-Xaa8; or of the theta defensin sequence Xaa1-Xaa2-Xaa3-Xaa4-Xaa5-Xaa1-Xaa6-Xaa7-Xaa8-Xaa1-Xaa2-Xaa6-Xaa4-Xaa5-Xaa1-Xaa3-Xaa7-Xaa8; or of the theta defensin sequence Xaa1-Xaa1-Xaa3-Xaa4-Xaa5-Xaa1-Xaa6-Xaa4-Xaa4-Xaa1-Xaa1-Xaa6-Xaa4-Xaa5-Xaa1-Xaa3-Xaa4-Xaa4, also can be chemically synthesized using routine methods of solid phase synthesis or can be expressed from a recombinant nucleic acid molecule encoding the theta defensin.

Additional exemplary theta defensins include a theta defensin comprising the amino acid sequence Arg-Cys-Ile-Cys-Thr-Arg-Gly-Phe-Cys (SEQ ID NO:) or Arg-Cys-Leu-Cys-Arg-Arg-Gly-Val-Cys (SEQ ID NO:). Another theta defensin analog comprises the amino acid sequence Xaa4-Xaa5-Xaa1-Xaa3-Xaa7-Xaa8-Xaa1-Xaa2-Xaa3, and still another theta defensin analog comprises the amino acid sequence Xaa4-Xaa5-Xaa1-Xaa6-Xaa4-Xaa4-Xaa1-Xaa1-Xaa6. Further exemplary theta defensins include peptides having the amino acid sequence Gly-Phe-Cys-Arg-Cys-Ile-Cys-Thr-Arg-Gly-Phe-Cys-Arg-Cys-Ile-Cys-Thr-Arg (SEQ ID NO:). The invention also provides a theta defensin having the amino acid sequence Gly-Val-Cys-Arg-Cys-Leu-Cys-Arg-Arg-Gly-Val-Cys-Arg-Cys-Leu-Cys-Arg-Arg (SEQ ID NO:).

As disclosed herein, the RTD1a, RTD1b, and RTD1c peptides can form heterodimers (RTD-1, -4, and -5) and homodimers (RTD-2, -3, and -6 see FIG. 1). Similarly, an analog of the RTD1a peptide, such as Xaa4-Xaa5-Xaa1-Xaa3-Xaa7-Xaa8-Xaa1-Xaa2-Xaa3, and an analog of the RTD1b peptide, such as Xaa4-Xaa5-Xaa1-Xaa6-Xaa4-Xaa4-Xaa1-Xaa1-Xaa6, can form heterodimers and homodimers. Such heterodimers and homodimers are theta defensin analogs of the invention. The dimers can be linked by a peptide bond and contain intrachain disulfide crosslinks (FIG. 1).

In general, a precursor theta defensin is obtained following chemical synthesis of the peptide, since the newly synthesized peptide is not cyclized and does not contain the appropriate intrachain crosslinking. Similarly, expression of a recombinant nucleic acid molecule encoding a theta defensin generally results in the production of a precursor theta defensin peptide, unless the peptide is expressed in a cell that can effect formation of the appropriate bonds. Accordingly, the term “precursor,” when used in reference to a theta defensin peptide, means a form of the peptide that lacks a peptide bond between the amino terminal and carboxyl terminal amino acids or lacks at least one of the three disulfide bonds characteristic of a theta defensin. Such precursor peptides can be converted into a mature cyclic theta defensin containing, for example, one, two or three disulfide bonds by exposing the precursor peptide to the appropriate conditions for effecting formation of the intrapeptide crosslinks (see U.S. Pat. Nos. 6,335,318 and 6,514,727; WO 00/68265; U.S. publications 20040014669 and 20030162718). However, precursor theta defensins also are contemplated as useful in the present invention so long as the precursor has antimicrobial activity or can be converted to an antimicrobial form. For example, acyclic forms of theta defensins have been found to have antimicrobial activity, in vitro and, therapeutically, in vivo as disclosed herein.

A theta defensin or theta defensin analog can be prepared by solid phase methods. A natural theta defensin can be modified to a form having a free amino or carboxyl terminus, which can optionally be amidated (see FIG. 1 and U.S. Pat. Nos. 6,335,318 and 6,514,727; WO 00/68265; U.S. publications 20040014669 and 20030162718). In addition, an analog can be generated by substituting one or more amino acids of a theta defensin, as desired, particularly by incorporating conservative amino acid substitutions. Such conservative amino acid substitutions are well known and include, for example, the substitution of an amino acid having a small hydrophobic side chain with another such amino acid (for example, Ala for Gly) or the substitution of one basic residue with another basic residue (for example, Lys for Arg). Similar conservative amino acid substitutions in other antimicrobial peptides such as indolicidin resulted in the production of indolicidin analogs that maintained their broad spectrum antimicrobial activity (see U.S. Pat. No. 5,547,939, issued Aug. 20, 1996, which is incorporated herein by reference). Thus, a theta defensin analog having, for example, a substitution of Leu-6 with a Val, Ile or Ala residue, or a substitution of Arg-8 or Arg-9 or Arg-13 or Arg-18 with a Lys residue similarly can be expected to maintain broad spectrum antimicrobial activity.

A theta defensin analog also can have substitutions of the cysteine residues involved in a disulfide bond with amino acids that can form an intrachain crosslink, for example, with tryptophan residues, which can form a di-tryptophan crosslink. Similarly to naturally occurring indolicidin, which is a linear antimicrobial peptide, indolicidin analogs having an intrachain di-tryptophan crosslink also have antimicrobial activity. Furthermore, substitution of the Trp residues involved in the di-tryptophan crosslink in an indolicidin analog with Cys residues results in an indolicidin analog that has an intrachain disulfide crosslink and exhibits broad spectrum antimicrobial activity. By analogy to such indolicidin analogs, a theta defensin analog can contain, in place of one or more of the characteristic disulfide bonds, one or more corresponding di-tryptophan, lactam or lanthionine crosslinks. For example, a crosslink in a theta defensin analog can be formed, for example, between two Trp residues, which form a di-tryptophan crosslink. In addition, a crosslink can be a monosulfide bond formed by a lanthionine residue. A crosslink also can be formed between other amino acid side chains, for example, a lactam crosslink formed by a transamidation reaction between the side chains of an acidic amino acid and a basic amino acid, such as between the γ-carboxyl group of Glu (or β-carboxyl group of Asp) and the ε-amino group of Lys; or can be a lactone produced, for example, by a crosslink between the hydroxy group of Ser and the γ-carboxyl group of Glu (or β-carboxyl group of Asp); or a covalent bond formed, for example, between two amino acids, one or both of which have a modified side chain.

A theta defensin peptide can also have the amino acid sequence Xaa1-Xaa2-Xaa9-Xaa4-Xaa10-Xaa1-Xaa11-Xaa4-Xaa4-Xaa1-Xaa1-Xaa12-Xaa4-Xaa13-Xaa1-Xaa14-Xaa7-Xaa8; or the amino acid sequence Xaa1-Xaa2-Xaa9-Xaa4-Xaa10-Xaa1-Xaa11-Xaa7-Xaa8-Xaa1-Xaa2-Xaa12-Xaa4-Xaa13-Xaa1-Xaa14-Xaa7-Xaa8; or the amino acid sequence Xaa1-Xaa1-Xaa9-Xaa4-Xaa10-Xaa1-Xaa11-Xaa4-Xaa4-Xaa1-Xaa1-Xaa12-Xaa4-Xaa13-Xaa1-Xaa14-Xaa4-Xaa4, wherein Xaa1 independently is an aliphatic amino acid such as Gly, Ile, Leu, Val or Ala; Xaa2 is an aromatic amino acid such as Phe, Trp or Tyr; Xaa4 independently is Arg or Lys; Xaa7 is Thr or Ser; Xaa8 is Arg or Lys; Xaa9 is Glu, Asp, Lys or Ser; Xaa10 is Glu, Asp, Lys or Ser; Xaa11 is Glu, Asp, Lys or Ser; Xaa12 is Glu, Asp, Lys or Ser; Xaa13 is Glu, Asp, Lys or Ser; Xaa14 is Glu, Asp, Lys or Ser. In such a theta defensin peptide, an intrachain crosslink can be formed between two amino acids, Xaa9 and Xaa14; Xaa10 and Xaa13; or Xaa11 and Xaa12, which correspond to the same position as disulfide crosslinks in natural theta defensin. The intrachain crosslink can be, for example, a lactam or lactone. Such a theta defensin can be a linear peptide and can optionally be amidated at the C-terminus.

In theta defensin peptides having less than three crosslinks, as found in native theta defensin, the amino acids at the positions corresponding to the native crosslinks, for example, amino acids Xaa3, Xaa5 and Xaa6 in a theta defensin formula that includes RTD-1, can be modified (see amino acid positions 3, 5 and 6 in RTD-1 of FIG. 1). For example if positions Xaa3 are disulfide crosslinked, the amino acids at position Xaa5 and Xaa6 can be non cysteine residues, for example, a hydrophobic amino acid such as Tyr, Val, Ile, Leu, Met, Phe or Trp; a small amino acid such as Gly, Ser, Ala, or Thr; or a large polar amino acid such as Asn or Gln.

If desired, a theta defensin analog of the invention can have one or more amino acid deletions or additions relative to a known theta defensin sequence, again, by analogy to indolicidin analogs, which can have a carboxyl terminal amino acid deletion or as many as five amino terminal amino acid deletions, yet still maintain broad spectrum antimicrobial activity. Thus, theta defensin analogs having one or a few deletions or additions at selected positions in the theta defensin sequence maintain broad spectrum antimicrobial activity and, as such, are considered functional fragments of a natural theta defensin (see FIG. 1 and U.S. Pat. Nos. 6,335,318 and 6,514,727; WO 00/68265; U.S. publications 20040014669 and 20030162718). As used herein, a “functional fragment” when used in reference to a theta defensin is a portion of a theta defensin that still retains some or all of the antimicrobial activity of a theta defensin. The antimicrobial activity of a theta defensin analog, or a functional fragment thereof, containing one or more amino acid substitutions, deletions or additions as compared to reference sequence can be confirmed using assays as disclosed herein or otherwise known in the art. For example, a residue added to a theta defensin peptide or peptide analog can be a homoserine residue (see FIG. 1 and U.S. Pat. Nos. 6,335,318 and 6,514,727; WO 00/68265; U.S. publications 20040014669 and 20030162718).

As used herein, the term “amino acid” is used in its broadest sense to mean the naturally occurring amino acids as well as non-naturally occurring amino acids, including amino acid analogs. Thus, reference herein to an amino acid includes, for example, naturally occurring proteogenic (L)-amino acids, as well as (D)-amino acids, chemically modified amino acids such as amino acid analogs, naturally occurring non-proteogenic amino acids such as norleucine, and chemically synthesized compounds having properties known in the art to be characteristic of an amino acid. As used herein, the term “proteogenic” indicates that the amino acid can be incorporated into a protein in a cell through a metabolic pathway.

Theta defensins can be chemically synthesized as a linear precursor peptide using solid phase Fmoc chemistry (see U.S. Pat. Nos. 6,335,318 and 6,514,727; WO 00/68265; U.S. publications 20040014669 and 20030162718). The linear peptide can be subjected to reducing conditions, then oxidized to allow formation of the disulfide bonds, and treated with ethylenediaminecarbodiimide to cyclize the peptide. The synthesized cyclic theta defensin can be characterized by reverse phase-high performance liquid chromatography (RP-HPLC), MALDI-TOF mass spectrometry and circular dichroism (CD) and comigrated with native theta defensin by acid-urea PAGE (see U.S. Pat. Nos. 6,335,318 and 6,514,727; WO 00/68265; U.S. publications 20040014669 and 20030162718).

Methods for synthesizing a theta defensin or theta defensin analog are well known to those skilled in the art (U.S. Pat. Nos. 6,335,318 and 6,514,727; WO 00/68265; U.S. publications 20040014669 and 20030162718; Tang et al., Science 286:498-502 (1999)). A linear peptide of an amino acid sequence corresponding to the amino acid sequence of theta defensin or an analog thereof can be synthesized. One or more crosslink bonds within the linear peptide can be formed, and the peptide cyclized by linking the carboxyl and amino termini to form a cyclic peptide. The crosslink formed can be a disulfide, lanthionine, lactam or lactone. The cysteine residues used in the linear peptide can be in a pre-formed activated ester form. If a disulfide crosslink is formed between two cysteines, the crosslink can be formed by oxidation. The formation of a peptide bond between the amino and carboxyl termini can be advantageously mediated by placing the carboxyl terminus and amino terminus of the linear peptide each approximately the same number of amino acids from the nearest cysteine.

An advantage of using chemical synthesis to prepare a theta defensin or theta defensin analog is that (D)-amino acids can be substituted for (L)-amino acids, if desired. The incorporation of one or more (D)-amino acids into a theta defensin analog can confer, for example, additional stability of the peptide in vitro or, particularly, in vivo, since endogenous endoproteases generally are ineffective against peptides containing (D)-amino acids. Naturally occurring antimicrobial peptides that have been chemically synthesized to contain (D)-amino acids maintain their antimicrobial activity (Wade et al., Proc. Natl. Acad. Sci. USA 87:4761-4765 (1990), which is incorporated herein by reference).

If desired, the reactive side group of one or more amino acids in a theta defensin or theta defensin analog can be modified or amino acid derivatives can be incorporated into the peptide (see, for example, Protein Engineering: A practical approach (IRL Press 1992); Bodanszky, Principles of Peptide Synthesis (Springer-Verlag 1984), each of which is incorporated herein by reference). Selective modification of a reactive group, other than those involved in formation of the three intrachain crosslinks characteristic of a defensin, can impart desirable characteristics upon a theta defensin analog, although modifications that allow the formation of intrachain crosslinks at the appropriate positions also can be effected. The choice of including such a modification is determined, in part, by the characteristics required of the peptide. Such modifications can result, for example, in theta defensin analogs having greater antimicrobial selectivity or potency than naturally occurring theta defensin. For example, a theta defensin analog having a free carboxyl terminus can be modified so that the C-terminus is amidated (see FIG. 1). Similarly, a theta defensin analog having a free amino terminus can be modified so that the N-terminus is acetylated.

The theta defensins are polypeptides having antimicrobial activity. As used herein, the term “polypeptide” when used in reference to a theta defensin is intended to refer to a peptide or polypeptide of two or more amino acids. The term is similarly intended to refer to derivatives, analogues and functional mimetics thereof. For example, derivatives can include chemical modifications of the polypeptide such as alkylation, acylation, carbamoylation, iodination, or any modification which derivatizes the polypeptide. Analogues can include modified amino acids, for example, hydroxyproline or carboxyglutamate, and can include amino acids that are not linked by peptide bonds. Mimetics encompass chemicals containing chemical moieties that mimic the function of the polypeptide. For example, if a polypeptide contains two charged chemical moieties having functional activity, a mimetic places two charged chemical moieties in a spatial orientation and constrained structure so that the charged chemical function is maintained in three-dimensional space. Thus, a mimetic, which orients functional groups that provide the antimicrobial function of a theta defensin, are included within the meaning of a theta defensin derivative. All of these modifications are included within the term “polypeptide” so long as the polypeptide retains its antimicrobial function as a theta defensin.

A theta defensin can incorporate polypeptide derivatives. Peptide derivatives are well known in the art (see, for example, U.S. Pat. No. 5,804,558, issued Sep. 8, 1998). For example, certain commonly encountered amino acids, which are not encoded by the genetic code, include, for example, beta-alanine (beta-Ala), or other omega-amino acids, such as 3-aminopropionic, 2,3-diaminopropionic (2,3-diaP), 4-aminobutyric and so forth, alpha-aminisobutyric acid (Aib), sarcosine (Sar), ornithine (Orn), citrulline (Cit), t-butylalanine (t-BuA), t-butylglycine (t-BuG), N-methylisoleucine (N-MeIle), phenylglycine (Phg), and cyclohexylalanine (Cha), norleucine (Nle), 2-naphthylalanine (2-Nal); 1,2,3,4-tetrahydroisoquinoline-3-carboxylic acid (Tic); β-2-thienylalanine (Thi); methionine sulfoxide (MSO); and homoarginine (Har).

In a theta defensin peptide or peptide analog thereof, one or more amide linkages (—CO—NH—) can be replaced with another linkage which is an isostere such as —CH₂NH—, —CH₂S—, —CH₂CH₂, —CH══CH—(cis and trans), —COCH₂—, —CH(OH)CH₂— and —CH₂SO—. This replacement can be made by methods known in the art (see, for example, Spatola, Vega Data Vol. 1, Issue 3, (1983); Spatola, in Chemistry and Biochemistry of Amino Acids Peptides and Proteins, Weinstein, ed., Marcel Dekker, New York, p. 267 (1983); Morley, J. S., Trends Pharm. Sci. pp. 463-468 (1980); Hudson et al., Int. J. Pept. Prot. Res. 14:177-185 (1979); Spatola et al., Life Sci. 38:1243-1249 (1986); Hann, J. Chem. Soc. Perkin Trans. I 307-314 (1982); Almquist et al., J. Med. Chem. 23:1392-1398 (1980); Jennings-White et al., Tetrahedron Lett. 23:2533 (1982); Szelke et al., EP 45665 (1982); Holladay et al., Tetrahedron Lett. 24:4401-4404 (1983); and Hruby, Life Sci. 31:189-199 (1982)).

In addition to polypeptide derivatives of a theta defensin, a chemical mimetic of a theta defensin peptide can be used. As described above, mimetics contain chemical functional groups that mimic the function of a theta defensin. Such a mimetic chemical can orient functional groups on a theta defensin peptide sufficient for antimicrobial activity. A mimetic places the functional chemical moieties in a spatial orientation and constrained structure so that the chemical function is maintained in three-dimensional space. Thus, a mimetic orients chemical functional groups that provide the theta defensin function of antimicrobial activity in an orientation that mimics the structure of a theta defensin.

A molecular model of a theta defensin has been previously described (see U.S. Pat. No. 6,335,318, issued Jan. 1, 2002; WO 00/68265). Using the molecular model of theta defensin, one skilled in the art can identify a chemical such as a peptidomimetic. As used herein, the term “peptidomimetic” is used broadly to mean a peptide-like molecule that has a similar structure and activity as a theta defensin. With respect to theta defensin peptides, peptidomimetics, which include chemically modified peptides, peptide-like molecules containing non-naturally occurring amino acids, peptoids and the like, have the antimicrobial activity upon which the peptidomimetic is derived (see, for example, “Burger's Medicinal Chemistry and Drug Discovery” 5th ed., vols. 1 to 3 (ed. M. E. Wolff; Wiley Interscience 1995)). Peptidomimetics can provide various advantages over a peptide, including that a peptidomimetic can be more stable during passage through the digestive tract and, therefore, useful for oral administration.

Methods for identifying a peptidomimetic are well known in the art and include, for example, the screening of databases that contain libraries of potential peptidomimetics. For example, the Cambridge Structural Database contains a collection of greater than 300,000 compounds that have known crystal structures (Allen et al., Acta Crystallogr. Section B, 35:2331 (1979)). This structural depository is continually updated as new crystal structures are determined and can be screened for compounds having suitable shapes, for example, the same shape as a theta defensin peptide. Another database, the Available Chemicals Directory (Molecular Design Limited, Information Systems; San Leandro Calif.), contains compounds that are commercially available and also can be searched to identify potential peptidomimetics of a theta defensin peptide.

As used herein, the term “antimicrobial selectivity” refers to the relative amount of antimicrobial activity of theta defensin, or a theta defensin analog, against a microorganism as compared to its activity against the environment to which it is administered, particularly its activity against normal cells in a treated individual. For example, a theta defensin analog that is characterized by having antimicrobial activity that is equivalent to native theta defensin, but having decreased hemolytic activity as compared to native theta defensin, is considered to have greater antimicrobial selectivity than native theta defensin.

As described previously and herein, naturally occurring theta defensins and analogs thereof have broad spectrum antimicrobial activity. As used herein, the term “broad spectrum,” when used in reference to the antimicrobial activity of theta defensin or an analog thereof, refers to the ability of the peptide to reduce or inhibit the survival or proliferative ability of various viruses, prokaryotic and eukaryotic microorganisms. For example, naturally occurring theta defensins and analogs thereof can exhibit antimicrobial activity against protozoans such as Giardia lamblia, Chlamydia sp. and Acanthamoeba sp.; viruses, particularly enveloped viruses such as herpes simplex virus and HIV-1; fungi such as Cryptococcus and Candida; various genera of gram negative and gram positive bacteria, including Escherichia, Salmonella and Staphylococcus and Listeria; and parasitic helminths such as liver flukes. Antimicrobial activity can occur through “microbicidal inhibition,” which refers to the ability of a theta defensin or theta defensin analog to reduce or inhibit the survival of a microorganism by killing or irreversibly damaging it, or through “microbistatic inhibition,” which refers to the ability of the theta defensin or theta defensin analog to reduce or inhibit the growth or proliferative ability of a target microorganism without necessarily killing it. Theta defensins are also effective therapeutically by virtue of their ability to neutralize microbial antigens or the effect of these antigens to produce an exaggerated inflammatory condition or to induce immune paralysis. Theta defensins and theta defensin analogs are also active in the presence of physiological salt and serum (see Example II and U.S. Pat. Nos. 6,335,318 and 6,514,727; WO 00/68265; U.S. publications 20040014669 and 20030162718).

A precursor theta defensin or theta defensin analog can be expressed from a recombinant nucleic acid molecule encoding the peptide. For example, a nucleic acid encoding a theta defensin peptide or precursor can be used to recombinantly express a theta defensin peptide or analog thereof. A nucleic acid molecule encoding a theta defensin can be chemically synthesized or can be cloned from a cell that contains a theta defensin gene or encodes a theta defensin mRNA, which can be converted to a cDNA. A nucleic acid molecule encoding a precursor theta defensin can be prepared by chemical synthesis, based on a known theta defensin amino acid sequence and knowledge in the art of codons encoding each amino acid.

In addition, a nucleic acid encoding a theta defensin analog having a free amino and carboxyl terminus can be synthesized and used to recombinantly express the theta defensin analog. Thus, a theta defensin analog can be expressed as a single, contiguous polypeptide without the need for trans splicing of component nonapeptides, as with the in vivo synthesis of native RTD-1-6.

RTD1 is encoded by two similar cDNAs, termed RTD1a and RTD1b, each of which contains 9 of the 18 amino acid residues in the mature RTD-1 peptide (see U.S. Pat. Nos. 6,335,318 and 6,514,727; WO 00/68265; U.S. publications 20040014669 and 20030162718). The cDNAs encode separate peptides, which become cyclized by formation of peptide bonds that join the two peptides.

A nucleic acid molecule encoding a precursor theta defensin or a theta defensin analog thereof can be cloned into an appropriate vector, particularly an expression vector, and the encoded peptide can be expressed in a host cell or using an in vitro transcription/translation reaction, thereby providing a means to obtain large amounts of the theta defensin. Optionally, the recombinant peptide can be produced as a fusion with a tag, such as a His tag, to facilitate identification and purification. Suitable vectors, host cells, in vitro transcription/translation systems, and tag sequences are well known in the art and commercially available.

An anti-theta defensin antibody can be used to substantially purify theta defensin from a sample. For example, a theta defensin antibody can be used to isolate naturally occurring theta defensin from leukocytes or from a cell expressing a recombinant nucleic acid molecule encoding a theta defensin or theta defensin analog.

A theta defensin or analog thereof having antimicrobial activity can be applied to an environment capable of sustaining the survival or growth of a microorganism or to an environment at risk of supporting such survival or growth, thus providing a means for reducing or inhibiting microbial growth or survival. Accordingly, a theta defensin or a theta defensin analog can be used to reduce or inhibit microbial growth by contacting an environment capable of sustaining microbial growth or survival with the antimicrobial peptide.

Theta defensins can be used to reduce or inhibit growth or survival of a microorganism in an environment capable of sustaining the growth or survival of the microorganism by administering an effective amount of a theta defensin analog to the environment, thereby reducing or inhibiting the growth or survival of the microorganism. As disclosed herein and described previously, theta defensins have broad spectrum antimicrobial activity. Theta defensins have also been found to be active in physiological salts and in serum (see Example II), indicating that the theta defensins can be effective at inhibiting microbial growth in an organism. Thus, theta defensins can be effective for treating polymicrobial infections, including sepsis.

As used herein, reference to “an environment capable of sustaining survival or growth of a microorganism” means a gaseous, liquid or solid material, including a living organism, in or upon which a microorganism can live or propagate. In view of the broad range of environments that allow the survival or growth of microorganisms as diverse, for example, as viruses, bacteria, fungi, protozoans and helminths, and further in view of the disclosed effectiveness of a theta defensin and theta defensin analogs against a broad spectrum of such microorganisms, the range of such environments that can be treated using a theta defensin or theta defensin analog includes, for example, a tissue or bodily fluid of an organism such as a human; a liquid such as water or an aqueous solution such as contact lens solution or eyewash solution; a food such as a food crop, a food product or a food extract; and an object such as the surface of an instrument used, for example, to prepare food or to perform surgery; and a gas such as that used for anesthetization in preparation for surgery. As described herein, theta defensins are particularly effective for treating polymicrobial peritonitis as shown using a mouse model of sepsis.

A method of the invention encompasses administering to the environment an effective amount of a theta defensin analog of the invention such that the antimicrobial peptide can contact a microorganism in the environment, thereby reducing or inhibiting the ability of the microorganism to grow or survive. A theta defensin analog can be used in a variety of procedures for reducing or inhibiting the survival or growth of microorganisms, including the microbicidal inhibition of survival of a microorganism as well as the microbistatic inhibition of growth. As such, a theta defensin analog can be used, for example, as a therapeutic agent, a food preservative, a disinfectant or a medicament. A theta defensin is particularly useful as a medicament to treat an infection in an individual, including sepsis.

A theta defensin analog can be particularly useful as a therapeutic agent for treating a patient suffering from a bacterial, viral, fungal or other infection due to a microorganism susceptible to the antimicrobial activity of the theta defensin or theta defensin analog. For example, a cyclic form of a theta defensin can be used since a cyclic theta defensin is particularly resistant to the activity of endogenous proteases and peptidases. Acyclic theta defensins have also been found to be active (see Example III). Similarly, modified forms of a theta defensin such as the analogs disclosed herein can be resistant to protease digestion. Thus, a theta defensin analog can be used to treat an individual suffering from a pathology caused, at least in part, by microbial infection, by administering a theta defensin or theta defensin analog to the individual under conditions that allow the theta defensin or analog thereof to contact the infecting microorganisms, thereby reducing or inhibiting the survival or growth of the microorganism and alleviating the severity of the infection. In addition, the presence of the theta defensin or analog thereof may mitigate against immune-mediate pathology such as that obtained with a hyperinflammatory response or a subsequent immunoparalysis.

For use as a therapeutic agent, the theta defensin or theta defensin analog can be formulated with a pharmaceutically acceptable carrier to produce a pharmaceutical composition, which can be administered to the individual, which can be a human or other mammal. A pharmaceutically acceptable carrier can be, for example, water, sodium phosphate buffer, phosphate buffered saline, normal saline or Ringer's solution or other physiologically buffered saline, or other solvent or vehicle such as a glycol, glycerol, an oil such as olive oil or an injectable organic ester.

A pharmaceutically acceptable carrier can contain physiologically acceptable compounds that act, for example, to stabilize or increase the absorption of the theta defensin or analog thereof. Such physiologically acceptable compounds include, for example, carbohydrates such as glucose, sucrose or dextrans; antioxidants such as ascorbic acid or glutathione; chelating agents such as ethylenediamine tetraacetic acid (EDTA), which disrupts microbial membranes; divalent metal ions such as calcium or magnesium; low molecular weight proteins; or other stabilizers or excipients. One skilled in the art would know that the choice of a pharmaceutically acceptable carrier, including a physiologically acceptable compound, depends, for example, on the route of administration of the composition.

A pharmaceutical composition containing a theta defensin or analog thereof can be administered to an individual by various routes, including by intravenous, subcutaneous, intramuscular, intrathecal or intraperitoneal injection; orally, as an aerosol spray; as an inhalant, or by intubation. If desired, the theta defensin or theta defensin analog can be incorporated into a liposome, a non-liposome lipid complex, or other polymer matrix, which further can have incorporated therein, for example, a second drug useful for treating the individual. Use, for example, of an antimicrobial indolicidin peptide incorporated into liposomes has been demonstrated to have antifungal activity in vivo (Ahmad et al., Biochem. Biophys. Acta 1237:109-114 (1995), which is incorporated herein by reference). Liposomes, which consist of phospholipids or other lipids, are nontoxic, physiologically acceptable and metabolizable carriers that are relatively simple to make and administer (Gregoriadis, Liposome Technology, Vol. 1 (CRC Press, Boca Raton Fla., 1984), which is incorporated herein by reference). The skilled artisan will select a particular route and method of administration based, for example, on the location of a microorganism in a subject, the particular characteristics of the microorganism, and the specific theta defensin or theta defensin analog that is administered.

In order to exhibit antimicrobial activity in an environment, an effective amount of a theta defensin analog is administered to the environment, and/or that which mitigates against immune-mediated pathology. As used herein, the term “effective amount” refers to the amount of a theta defensin analog that reduces or inhibits the survival or growth of a microorganism in an environment. In particular, an effective amount of a theta defensin analog produces only minimal effects against the environment, although the level of an acceptable deleterious effect is weighed against the benefit caused by the antimicrobial and/or immuno-modulating effects. One skilled in the art can readily determine an effective amount by administering various amounts of a theta defensin and determining the antimicrobial therapeutic effectiveness of the theta defensin.

A theta defensin analog can be administered to a subject such as a human systemically at a dose ranging, for example, from about 1 to about 100 mg/kg body weight, for example, at a dose of about 10 to about 80 mg/kg, particularly about 10 to about 50 mg/kg. A theta defensin can be administered, for example, at a dose of about 0.01 mg/kg, about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12 mg/kg, about 15 mg/kg, about 20 mg/kg, about 25 mg/kg, about 30 mg/kg, about 40 mg/kg, about 50 mg/kg, about 60 mg/kg, about 70 mg/kg, about 80 mg/kg, about 90 mg/kg, or about 100 mg/kg. A theta defensin analog also can be incorporated into liposomes, if desired, in which case the total amount administered to a subject generally can be reduced. Furthermore, a theta defensin analog can be administered orally to a subject at a dose ranging from about 1 to 100 mg/kg body weight, for example at a dose of about 10 to 200 mg/kg, in particular about 20 to 100 mg/kg, or in the amounts described above. In addition, a theta defensin analog can be administered topically to an environment, which can be a human subject, or can be placed in a solution, at a concentration, for example, of about 0.1 to 10 mg/ml, for example, at a concentration of about 0.5 to 5 mg/ml. A theta defensin can be administered topically, for example, at a concentration of about 0.01 mg/ml, about 0.05 mg/ml, about 0.1 mg/ml, about 0.2 mg/ml, about 0.5 mg/ml, about 0.7 mg/ml, about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 15 mg/ml or about 20 mg/ml. Although theta defensins generally are effective in vitro at microgram per ml amounts, an effective amount for administration to a particular environment will depend, in part, on the environment. For example, when administered to a mammal such as a human, a theta defensin analog, in addition to having antimicrobial activity, can have an undesirable side effect. The skilled artisan will recognize that the level of such side effects must be considered in prescribing a treatment and must be monitored during the treatment period, and will adjust the amount of the theta defensin analog that is administered accordingly. It is understood that veterinary uses of theta defensins are also contemplated, for example, in treating polymicrobial infections or sepsis.

An effective amount of a theta defensin analog also will vary depending, for example, on the characteristics of the target microorganism, the extent of prior infection or growth and the specific theta defensin analog that is administered. In addition, an effective amount depends on the form in which the theta defensin is administered. For example, incorporation of another antimicrobial peptide, indolicidin, into liposomes allowed administration of a higher amount of the peptide than “free” indolicidin, without producing unacceptable side effects, such that fungal infection in mice could be cured (Ahmad et al., supra, 1995).

A theta defensin can also be administered in combination with other antimicrobial agents, for example, antibiotics. The effective amount of a theta defensin to be administered to an individual can be adjusted if the theta defensin is administered in combination with another antimicrobial compound such as an antibiotic. Thus, one skilled in the art can adjust the dosage of the theta defensin so that the combination of the theta defensin and other antimicrobial therapy is optimally effective for inhibiting the growth or survival of a microorganism in an environment. Antibiotics are often given in combination therapy, for example, using a combination of antibiotics effective against gram positive, gram negative bacteria and anaerobes. An exemplary combination of antibiotics frequently used to treat sepsis includes cephalosporin, gentamicin, and metronidizole. A theta defensin can be administered in combination with a single antibiotic or multiple antibiotics, as desired. Thus, the theta defensin peptides of the invention can be administered in combination with known antibiotics such as aminoglycosides, cephalosporins, macrolides, erythromycin, monobactams, penicillins, quinolones, sulfonamides, tetracycline, and various anti-infective agents. Those skilled in the art can refer to the Physician's Desk Reference, 50th Ed (Medical Economics (1996)), or similar reference manuals for a more complete listing of known antibiotics which could be used in combination with the inventive phage preparations. For example, a theta defensin effective against various strains of staphylococcus could be used in combination with a cephalosporin such as Keflex™ or Keftab™. Those skilled in the art, using the guidelines provided herein, are capable of designing an effective treatment regimen by either using the theta defensin peptides alone or in combination with antibiotics.

A theta defensin can also be combined with other forms of antimicrobial therapy, including therapies for treatment of sepsis, for example, recombinant human activated protein C; corticosteroid therapy; early, goal-directed resuscitation therapy; modulation of heparin, tissue factor pathway inhibitor and high mobility group protein; intensive insulin therapy for hyperglycemia, interleukin 12, antibodies against C5a, antibodies to macrophage migration inhibitory factor, blocking apoptosis, administration of poly-ADP-ribose polymerase 1 (PARP), stimulation of the vagus nerve; nicotine (see, for example, Rice and Bernard, Ann. Rev. Med. 56:225-248 (2005) (published online August 2004 in Review in Advance); Hotchkiss and Karl, NEJM 348:138-150 (2003), Wang et al., Nat. Med. November: 10(11) 1216-21 (2004), each of which is incorporated herein by reference).

Inflammation is a physiologic response to a variety of stimuli such as infections and tissue injury. Neutrophils are the predominant cell type infiltrating an area of inflammation in the early stages of an inflammatory response. A variety of inflammatory mediators are released that serve to trigger or enhance specific aspects of the inflammatory response, including chemokines, lipids such as arachidonic acid, prostaglandins and leukotrienes, and cytokines. For example, as shown previously (U.S. publication 20040014669), lipopolysaccharide (LPS) was used as a model of inflammation and was found to stimulate the production of several cytokines including tumor necrosis factor-α, several interleukins (IL-1β, 2, 5, 6, 7, and 10), several chemokines (MIP-1-δ, RANTES) and growth stimulatory factors (GM-CSF, SCF, and TGF-β1). The addition of theta defensins reduced the levels of many cytokines that are released by LPS-stimulated cells, indicating that theta defensins can play a role during an inflammatory response. Reduction of pro-inflammatory cytokines such as TNF-α and IL-1β by RTD-1 indicates the anti-inflammatory property of the peptide are mediated through the regulation of cytokine production. Accordingly, theta defensins and analogs thereof can be used to decrease or inhibit the expression of pro-inflammatory molecules. Exemplary pro-inflammatory molecules include tumor necrosis factor-α, interleukin-1β (IL-1β), IL-2, IL-5, IL-6, IL-7, and IL-10, chemokines such as MIP-1-δ and RANTES, and growth stimulatory factors such as GM-CSF, SCF, and TGF-β1. Decreasing or inhibiting these or other signs of inflammation can be mediated by a theta defensin or analog thereof.

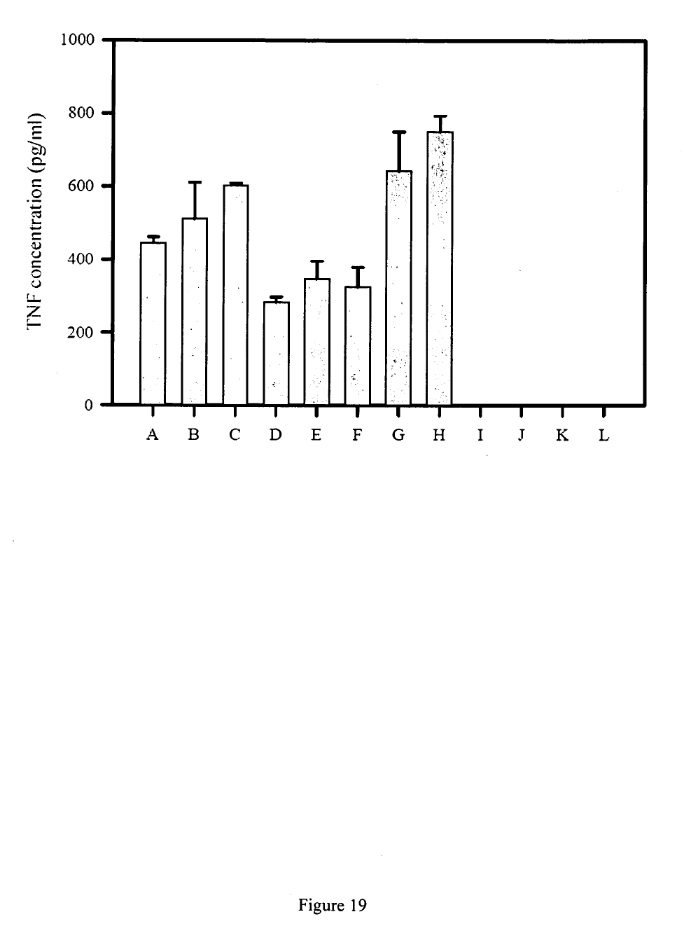

As disclosed herein, a theta defensin can modulate the release of TNFα by human PBMCs. Without being bound by a particular mechanism, theta defensins modulate cytokine production (see FIG. 19 and U.S. publication 20040014669). Therefore, the potent efficacy of theta defensins in treating polymicrobial peritonitis may be due to direct antimicrobial activity as well as modulating inflammatory responses associated with a condition such as sepsis. As used herein, decreasing an inflammatory response refers to a decrease of one or more responses associated with inflammation.

The invention provides a method of inhibiting undesirable microbial growth in an individual by administering to the individual an effective amount of a theta defensin, whereby microbial growth is inhibited. As used herein, “undesirable microbial growth” refers to microbial growth that is not within the location or amount of growth within a normal diversity of flora. For example, growth of a microorganism in a location in which that particular organism does not normally grow or in a location that is usually sterile is included within the meaning of an undesirable microbial growth. Undesirable microbial growth can include an infection, in which microogranisms that are not typically a component of the normal flora multiply within or on the body. Polymicrobial peritonitis is an example of undesirable microbial contamination of normally sterile tissue space usually leading to systemic inflammatory response syndrome, sepsis, and commonly septic shock. The organisms involved in such a process are a complex mixture of gram positive, gram negative, aerobic and anaerobic bacteria, many of which are not identifiable due to the fact that they cannot be cultured in vitro.

The invention additionally provides a method of inhibiting a deleterious effect of microbial growth in an individual by administering to an individual an effective amount of a theta defensin, whereby microbial growth is inhibited. An exemplary deleterious effect is an or hyper-inflammatory response, or a subsequent state of immunosuppression or immunoparalysis. As disclosed herein, theta defensins modulate cytokine production and can therefore modulate an inflammatory response. Exemplary deleterious effects include autoimmune reactions, adult respiratory distress syndrome, coagulation disorders, and multiple organ dysfunction. Theta defensins can be used to inhibit microbial growth associated with a deleterious effect such as autoimmune disease or cancer.

The methods of the invention can be used with a variety of theta defensins including, for example those shown in FIG. 1 and described previously (U.S. Pat. Nos. 6,335,318 and 6,514,727; WO 00/68265; U.S. publications 20040014669 and 20030162718).

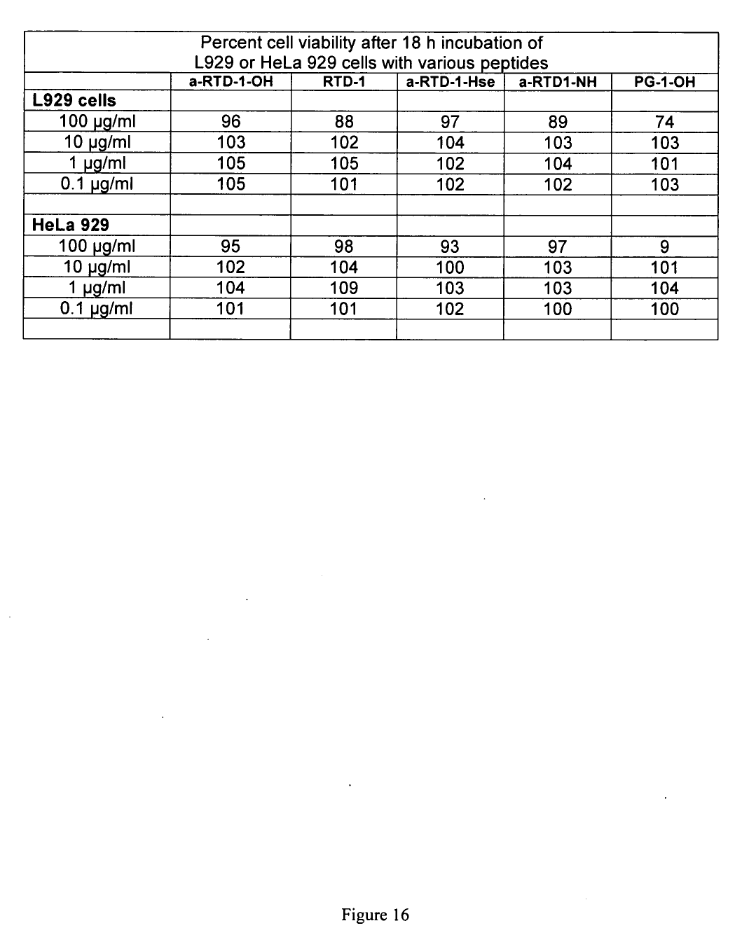

As disclosed herein, theta defensins are effective at inhibiting microbial growth and can be used to treat complex conditions such as sepsis. Previous studies have focused on the use of single organisms in a model to determine the activity of theta defensins against particular types of organisms. As disclosed herein, theta defensins have particularly potent activity against a wide range of organisms. For example, the polymicrobial peritonitis model of sepsis disclosed herein not only shows that theta defensins are effective against particular microorganisms but are also effective against a wide range of organisms and/or the immune mediated pathologic sequalae. Cecal ligation and perforation allows numerous types of intestinal flora to flourish in the peritoneum. Many of these organisms are not well characterized, since a large percentage of natural gut flora cannot be cultured. Nevertheless, theta defensins are effective at inhibiting the growth of these organisms and/or their immune-mediated pathologic effects, as evidenced by the dramatic increase in survival of mice treated with theta defensins in a mouse model of polymicrobial peritonitis (see Example VI). Furthermore, the theta defensins were adminstered 4 hours after perforation, allowing onset of a raging peritoneal infection. Therefore, the theta defensins are highly effective even significantly after onset of infection. Furthermore, theta defensins are advantageous in that they exhibit extremely low toxicity (see Example V and FIGS. 15-17). Theta defensins can therefore be administered after onset of infection, for example, 1 hour, 2 hours, 3 hours, 4 hours, 5 hours, 6 hours, 7 hours, 8 hours, 9 hours, 10 hours, 12 hours, 15 hours, 20 hours, 22 hours, 24 hours, 36 hours, 48 hours, or at any time determined by one skilled in the art to provide effective treatment.

As disclosed herein, acyclic versions of theta defensins were found to be effective in vivo. This result was unexpected given that acyclic versions of theta defensins are less active in vitro. For example, acyclic RTD-1 and acyclic RTD-2 were found to be as active as the corresponding cyclic peptides, in vivo. Furthermore, acyclic theta defensins have the advantage of being easier to generate since the acyclic peptides do not require cyclization. For example, acyclic theta defensins can be readily generated by recombinant expression, for example, in a multicopy construct since, once expressed, there is no need to cyclize the peptides.

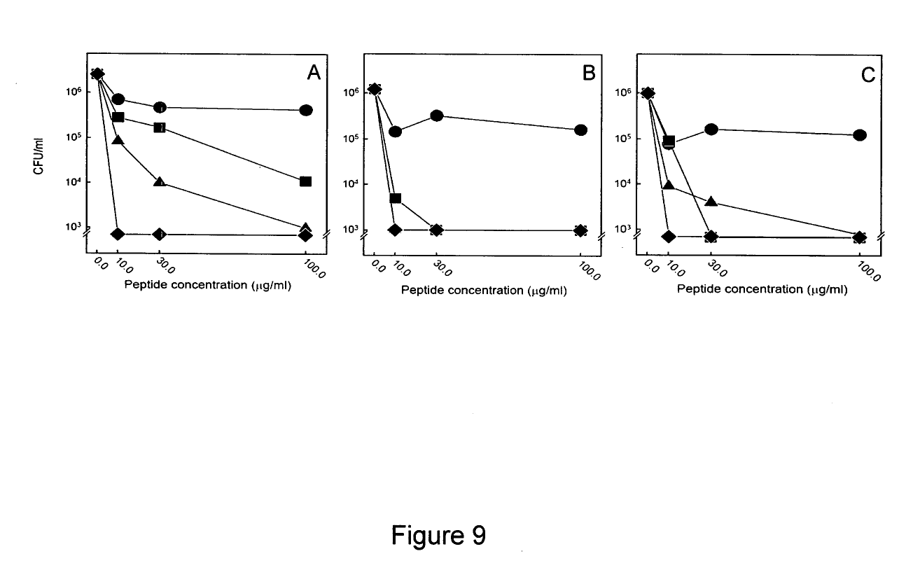

Additionally disclosed herein, theta defensins are active in salts and in serum (see Example II). This has not been found for all antimicrobial peptides. Furthermore, RTD-2 and RTD-3 were found to have higher antimicrobial activity than RTD-1 in the presence of salts (see FIG. 9). These findings were unexpected given that the structures of these peptides are similar. Also, the activities of RTD-1, 2 and 3 in vitro under low salt conditions are nearly indistinguishable in the absence of physiologic salt. Therefore, RTD-2 and RTD-3, or analogs thereof, are particularly useful for administration under physiological conditions. As shown in FIG. 9, RTD-2 and RTD-3 essentially have sterilizing activity, that is, give >99% microbicidal activity against Staphylococcus aureus.

As further disclosed herein, theta defensins also inhibit β-galactosidase activity. Without being bound by a particular mechanism, an additional activity of theta defensins can be enzyme inhibitory activity. Other enzymes can be inhibited by theta defensins similarly to β-galactosidase. Thus, a theta defensin can be used to inhibit an enzymatic activity. Such inhibitory activity could contribute to the high potency of the theta defensins. For example, theta defensins appear to exert their effect through more than a direct antimicrobial activity since the amount of theta defensin administered intravenously in the polymicrobial peritonitis model is unlikely to provide a sufficiently high enough concentration for a direct antimicrobial activity that is sterilizing in this environment. It is possible that other theta defensin activities in addition to direct antimicrobial activity, such as modulation of inflammation or enzyme activities, could contribute to the potency of theta defensins observed in a polymicrobial peritonits model (see Example VI).

The small size, stability, and cationicity of theta defensins allows for their interaction with macromolecules (e.g., cytokines, coagulation factors, cellular receptors) that pathologically amplify or suppress immune function in the setting of sepsis. This property modulates the pathologic inflammatory responses that occur during or following systemic microbial infection.

It is understood that modifications which do not substantially affect the activity of the various embodiments of this invention are also provided within the definition of the invention provided herein. Accordingly, the following examples are intended to illustrate but not limit the present invention.

### EXAMPLE I

### Antimicrobial Activity of Theta Defensin Compounds In Vitro

This example describes assay methods and antimicrobial properties of theta defensin compounds assayed in vitro.

The antimicrobial activities of various theta defensin compounds were measured using a diffusion assay against bacteria (Staphylococcus aureus 502a and Escherichia coli ML35) and fungi (Candida albicans 16820 and Cryptococcus neoformans 271A). The antimicrobial activities were determined in an agar diffusion assay as described previously (Osapay et al., J. Biol. Chem. 275:12017-12022 (2000)). Briefly, 10-μl wells were bored in a 9-cm² plate of agarose, buffered with 10 mM 1,4-piperazine-bis(ethanesulfonic acid) (PIPES), pH 7.4, containing 5 mM glucose, and seeded with 1×10⁶ mid-log phase cells. Five-μl aliquots of each peptide, dissolved in 0.01% acetic acid (HOAc) at 10-300 μg/ml, were added to each well. After incubation at 37° C. for 2 h, the seeded agar was overlaid with molten agarose containing 6% trypticase soy broth (for bacteria) or Sabouraud dextrose broth (for fungi). Plates were incubated at 37° C. for 18-24 h, and antimicrobial activity was determined by measuring the diameter of clearing around each well.

The microbicidal activities of various theta defensin peptides were determined by incubating 2×10⁶ CFU/ml with peptides (0-100 μg/ml) in 50 μl of low salt diluents: 10 mM PIPES buffer containing 5 mM glucose, pH 7.4, 10 mM Tris-HCl, pH 7.4, or 10 mM sodium phosphate, pH 7.4, or the same diluents supplemented with 25 to 154 mM NaCl. After 2 h incubation at 37° C., the cell suspensions were diluted 1:50 with 10 mM sodium phosphate buffer, pH 7.4, and exponentially spread with an Autoplate 400 (Spiral Biotech, MD) onto trypticase soy agar (bacteria) or Sabouraud dextrose agar (fungi). After incubation at 37° C. for 18-48 h, colonies were counted and cell survival was expressed as CFU/ml.

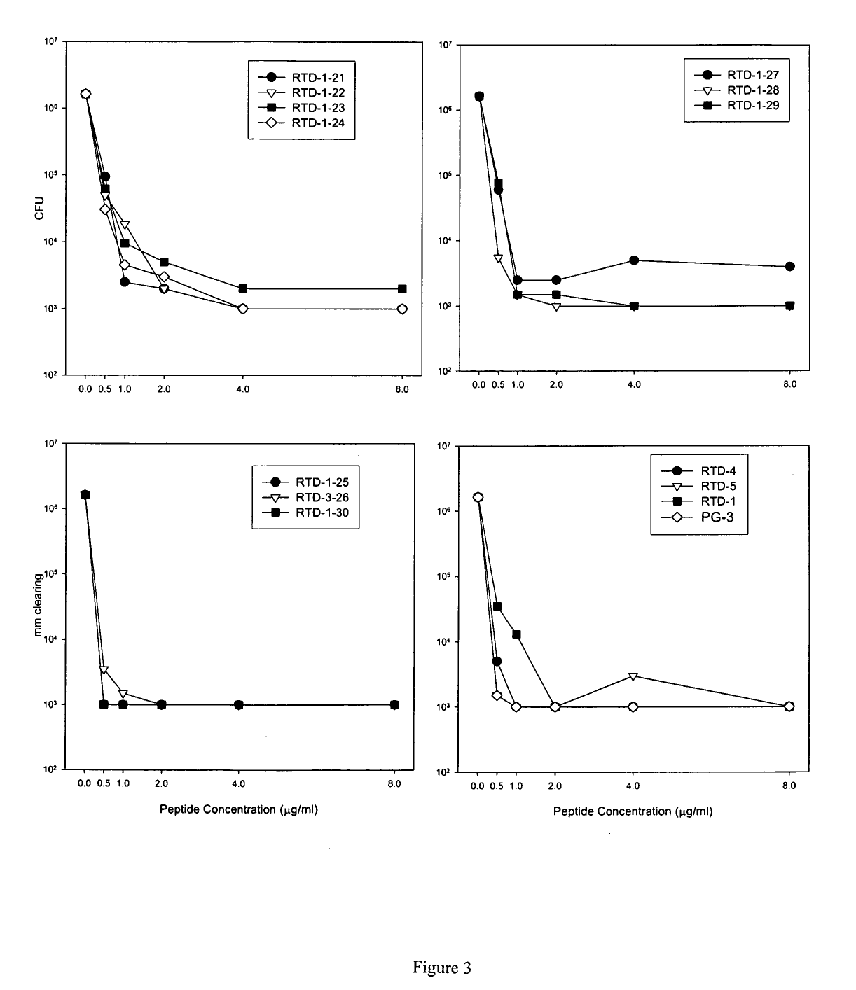

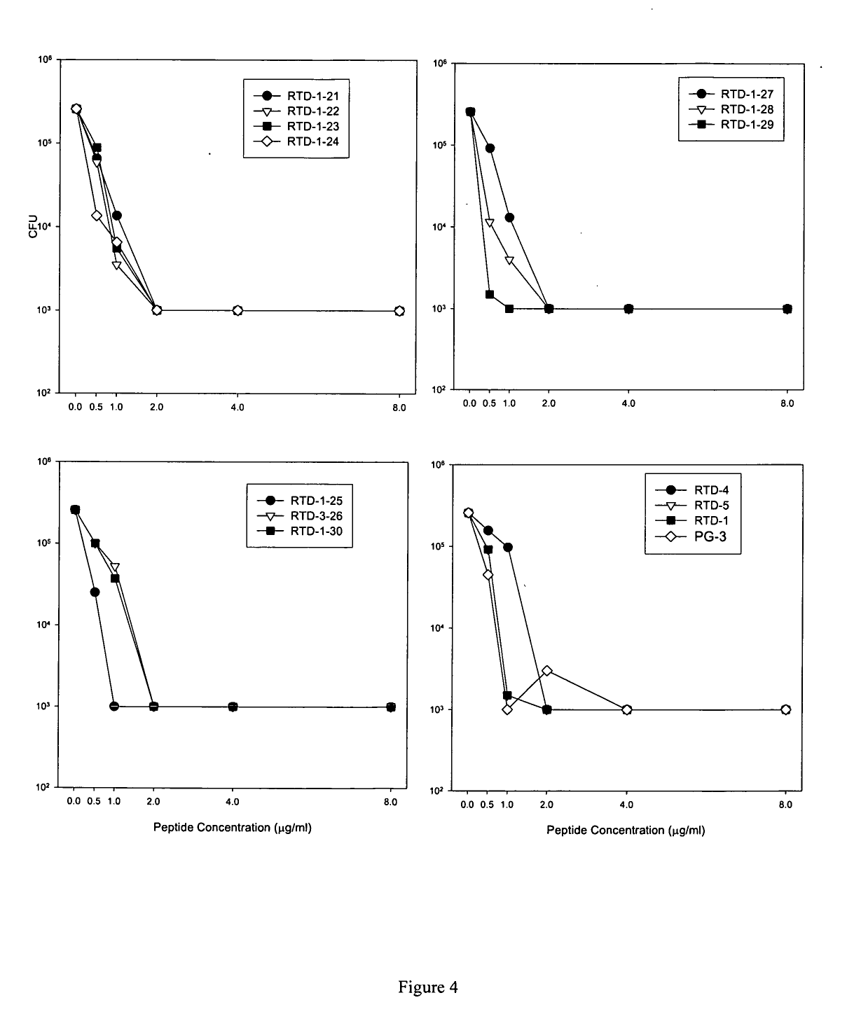

FIGS. 2-4 show the results of microbicidal assays performed with various theta defensins and protegrin. The corresponding structures of the peptides are shown in FIG. 1. Cells were incubated for 2 hours at 37° C. in 10 mM PIPES, pH 7.4, and 5 mM glucose, as described above. FIG. 2 shows the microbicidal activity of theta defensins against Escherichia coli ML35. FIG. 3 shows the microbicidal activity of theta defensins against Staphylococcus aureus 502A. FIG. 4 shows the microbicidal activity of theta defensins against Candida albicans.

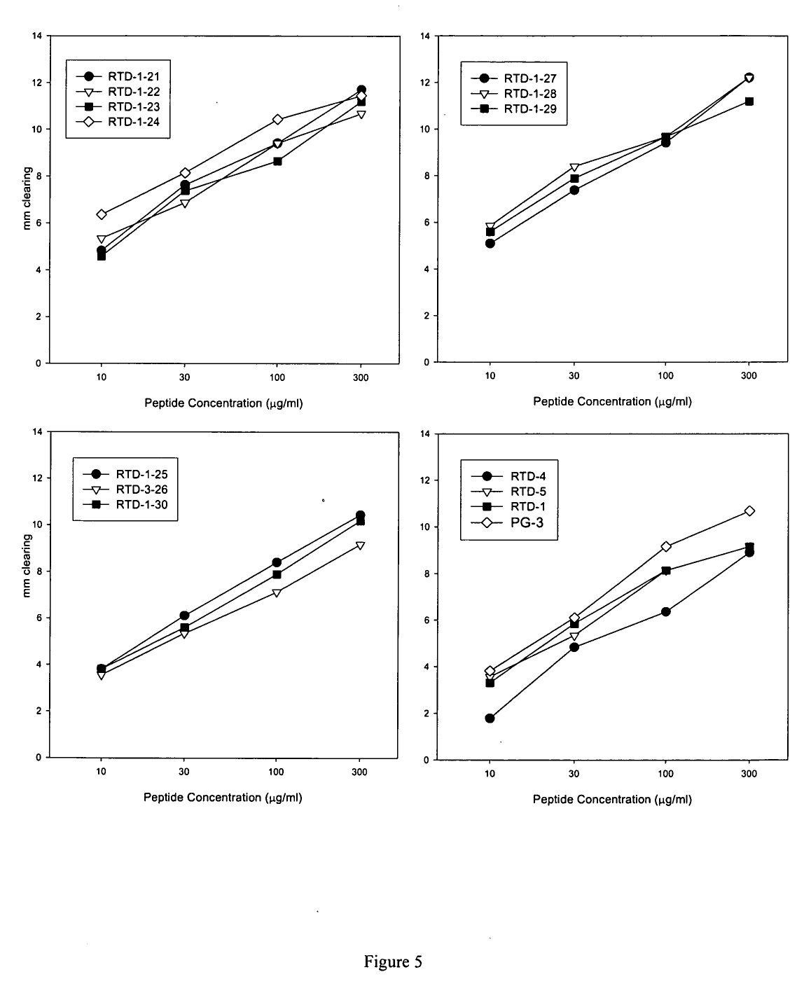

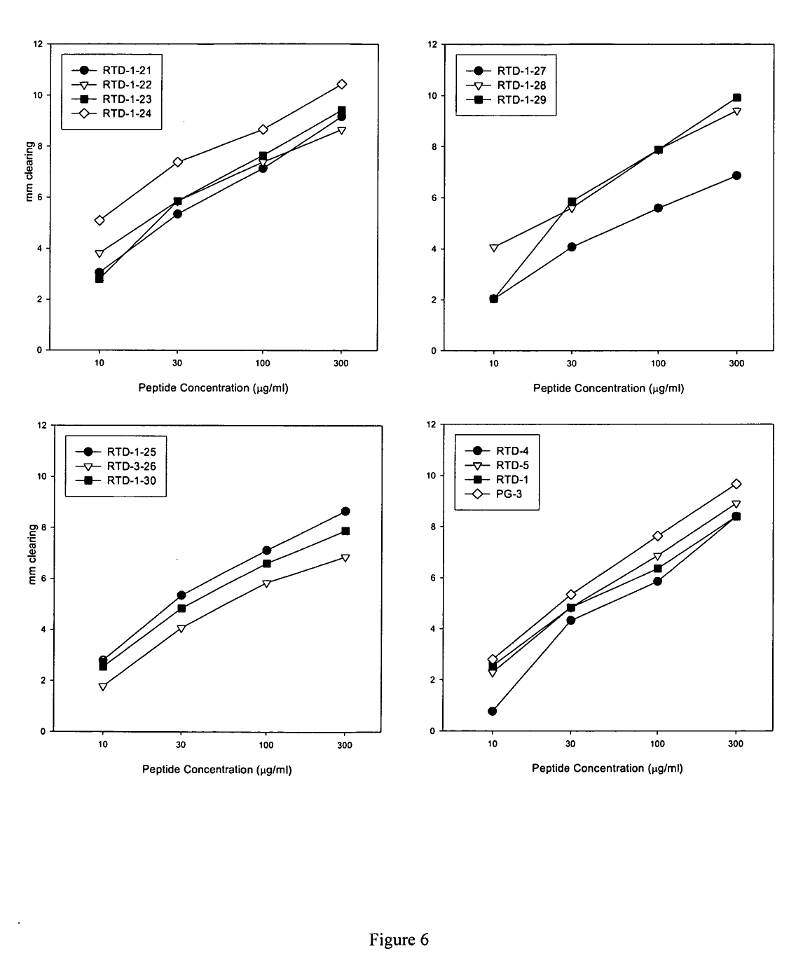

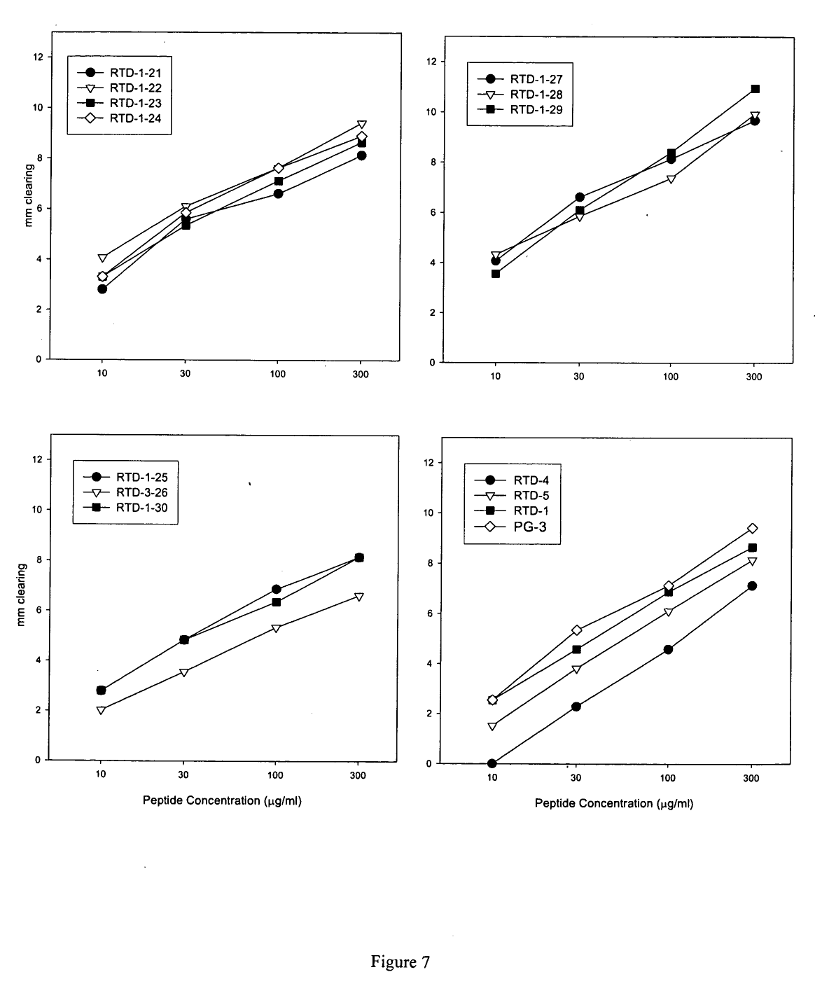

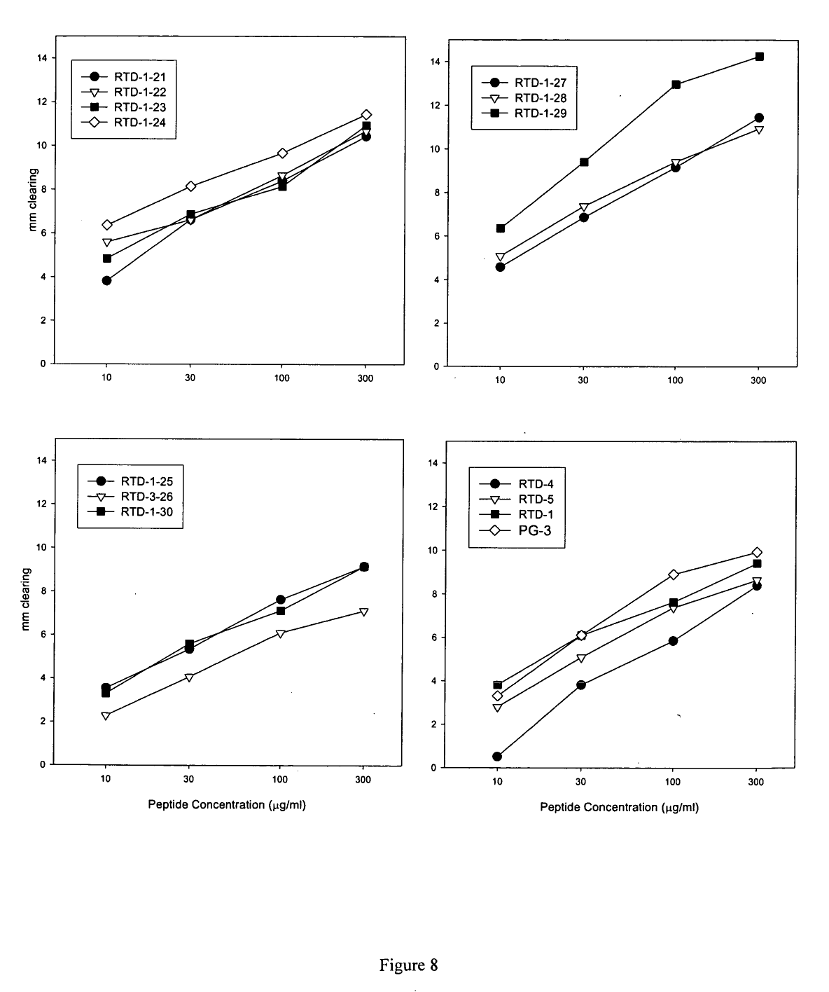

FIGS. 5-8 show antimicrobial activities of various theta defensins as measured using the diffusion assay. Cells were incubated for 2 hours at 37° C. in 10 mM PIPES, pH 7.4, and 0.03% glucose, as described above. FIG. 5 shows the antimicrobial activities of various theta defensins against E. coli ML35. FIG. 6 shows the antimicrobial activities of various theta defensins against S. aureus 502A. FIG. 7 shows the antimicrobial activities of various theta defensins against C. albicans. FIG. 8 shows the antimicrobial activities of various theta defensins against Cryptococcus neoformans 270A.

These results demonstrate that theta defensins exhibit broad spectrum antimicrobial activity, including against gram negative and gram positive bacteria as well as fungi.

### EXAMPLE II

### Antimicrobial Activity of Theta Defensins in Physiological Salts and Serum

This examples describes the antimicrobial activities of theta defensins in physiological salts and serum.

Staphylocidal activities of theta defensins and protegrin were assayed in various buffers and salt concentrations. Log-phase bacteria were incubated for 2 h at 37° C. with increasing concentrations of the peptides (FIG. 9). Cells were incubated with increasing concentrations of RTD-1 (filled circles), RTD-2 (filled triangles), RTD-3 (filled squares), and PG-1 (filled diamonds) in 10 mM PIPES (FIG. 9A), 10 mM Tris-Cl (FIG. 9B), or 10 mM sodium phosphate (FIG. 9C), at pH 7.4, containing 154 mM NaCl. As shown in FIG. 9, RTD-2 and RTD-3 exhibited more potent antimicrobial activity in the presence of salt compared to RTD-1, even though the structures of these theta defensins are very similar.

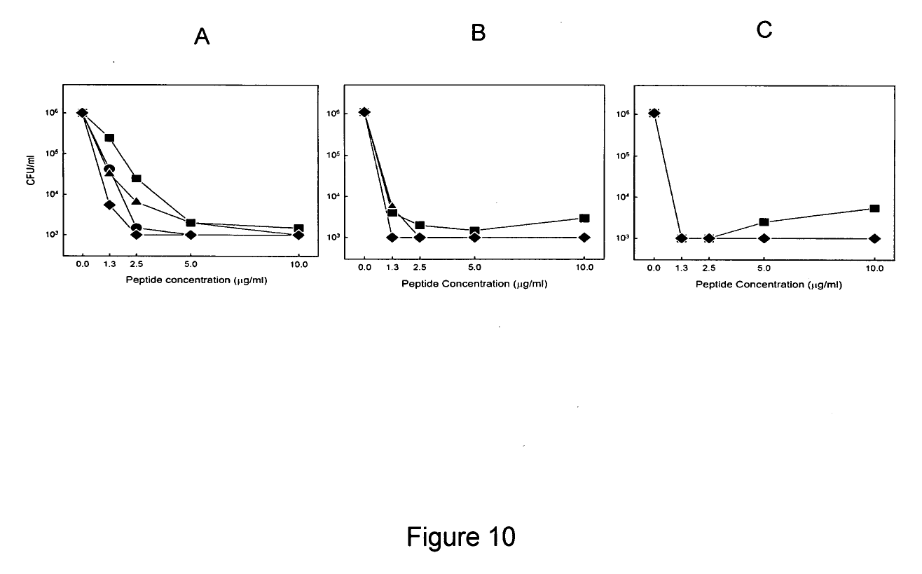

The bactericidal activities of theta defensins against E. coli in various physiological salt concentrations were also tested. FIG. 10 shows bactericidal activities of theta defensins and PG-1 in physiologic CaCl₂ and MgCl₂. Killing of E. coli ML35 was measured by incubating increasing concentrations of RTD-1 (filled circles), RTD-2 (filled triangles), RTD-3 (filled squares), and PG-1 (filled diamonds) with log-phase E. coli ML35 in 10 mM PIPES, pH 7.4, with CaCl₂ (1.2 mM) (FIG. 10A), MgCl₂ (0.8 mM) (FIG. 10B), or in control incubations where the divalent salts were omitted (FIG. 10C).

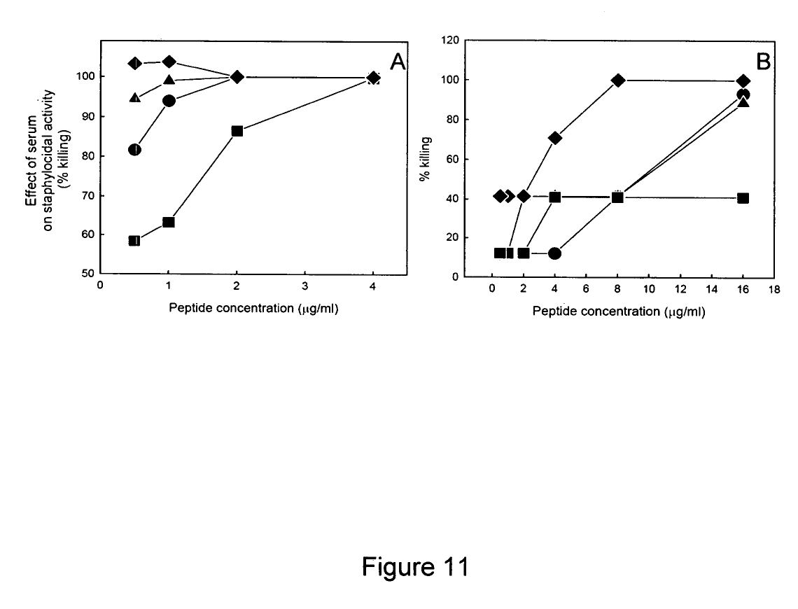

The activities of theta defensins in the presence of serum were also analyzed. FIG. 11 shows the bactericidal activities of theta defensins and PG-1 in serum. Dose-dependent bactericidal activities of RTD-1 (filled circles), RTD-2 (filled triangles), RTD-3 (filled squares), and PG-1 (filled diamonds) against bacteria were determined in 10 mM PIPES, pH 7.4, containing 10% normal (for S. aureus) or heat-inactivated (for E. coli) human serum. As shown in FIG. 11A, staphylocidal activities were determined by colony counting and expressed as percentages of killing relative to control incubations lacking serum. As shown in FIG. 11B, colicidal activities were expressed as percentages of killing relative to control incubations lacking peptides.

These results show that RTD-1, RTD-2 and RTD-3 are active in the presence of physiological salts and serum.

### EXAMPLE III

### Anti-HIV Activities of Theta Defensins

This example describes anti-retroviral activity of theta defensins against human immunodeficiency virus (HIV).

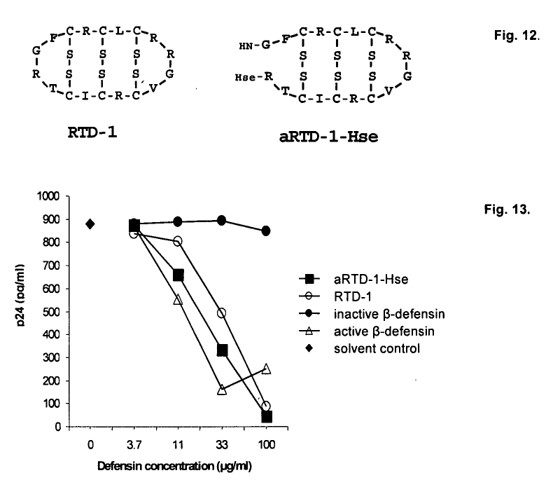

The activities of RTD-1 and acyclic-RTD-1-Hse against HIV were tested. FIG. 12 shows the structures of RTD-1 and acyclic-RTD-1-Hse (aRTD-1-Hse). Peptide aRTD-1-Hse was produced as described previously (see, for example, US 20040014669, which is incorporated herein by reference).

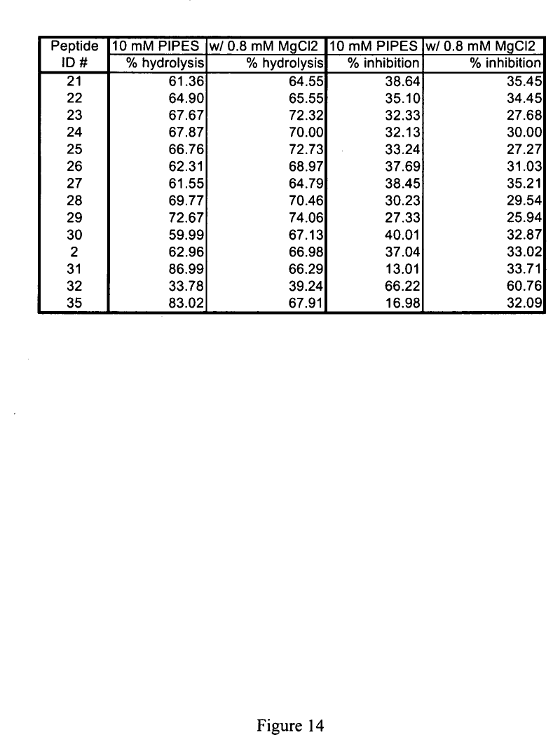

FIG. 13 shows anti-HIV activities of theta and beta defensins. Phytohaemagglutinin (PHA)-stimulated peripheral blood mononuclear cells (PBMCs), pre-incubated for 3 hours with serial dilutions of defensins, were mixed with 1000 TCID₅₀ (50% infectivity of susceptible cells in tissue culture) of a primary R5 strain of HIV-1. Two hours later, the cells were washed, and fresh medium containing defensin and 20 U/ml of IL-2 was added. Five days after infection, 50% of the medium was removed and replaced with fresh medium containing defensin and IL-2. On day 7 of infection, the levels of HIV protein p24 in supernatant fluid were measured by ELISA. The inactive β-defensin was a hybrid of human β-defensins 2 and 3. The active β-defensin was human β-defensin-3.

These results show that cyclic and acyclic theta defensins have anti-HIV activity.

### EXAMPLE IV

### Effect of Theta Defensins on Enzymatic Activity

This example describes the effect of theta defensins on β-galactosidase activity.

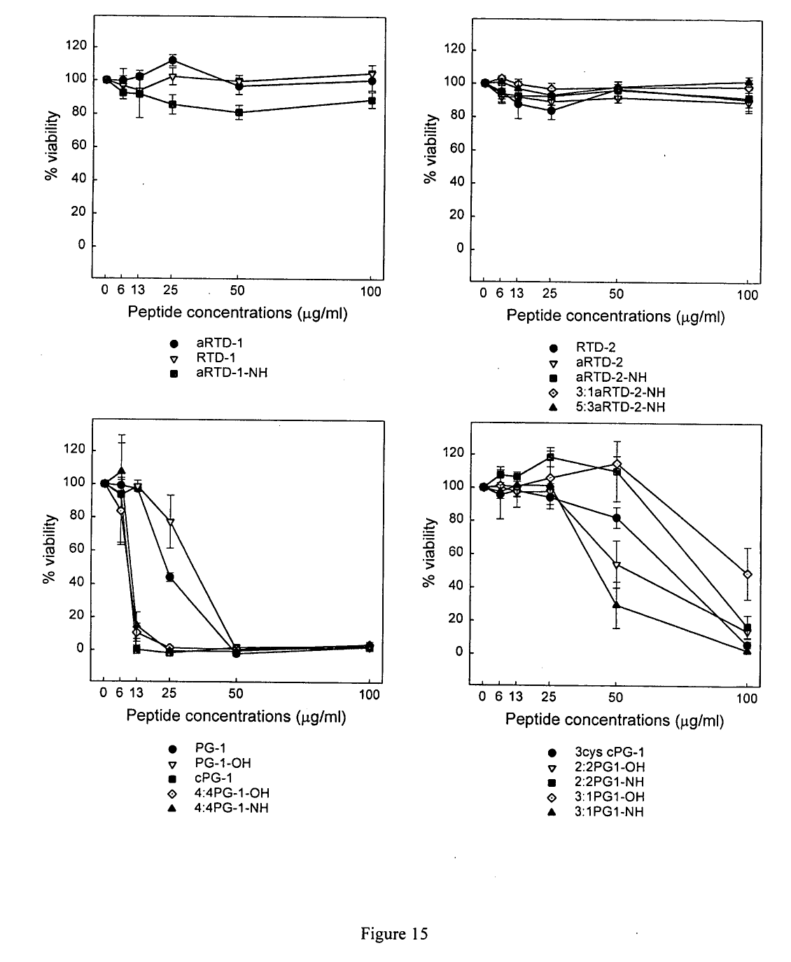

Purified β-galactosidase (3 nM) was incubated with 2 μg/ml of various theta defensin peptides, as indicated in FIG. 14. The peptide identification numbers listed correspond to the peptides shown in FIG. 1. The peptides were incubated in 10 mM PIPES, pH 7.4, with or without 0.8 mM MgCl₂, as indicated in FIG. 14. β-Galactosidase activity was measured as the rate of o-nitrophenyl-beta-D-galactopyranoside (ONPG) hydrolysis, and the activity with peptide was compared to that obtained in the absence of peptide. As shown in FIG. 14, various theta defensins inhibited β-galactosidase enzymatic activity.

These results demonstrate that theta defensins are potent enzyme inhibitors and that the inhibition is not overcome by addition of Mg⁺².

### EXAMPLE V

### Ex Vivo Toxicity Analysis of Theta Defensins

This example describes ex vivo toxicity studies with fibroblasts and red blood cells (RBCs).

The cytoxicity of various theta defensin peptides was tested in fibroblasts. The cytoxicity of each peptide, as shown in FIG. 15, was evaluated by determining the viability of HS68 fibroblasts (˜5×10³ cells per well) after 60 min exposure to 0-100 μg/ml of each peptide at 37° C. in Dulbeccos's modified Eagle medum (DMEM) containing 0.4% fetal bovine serum. These data demonstrate that theta defensins (top two panels) are much less cytotoxic than protegrins and protegrin analogs (bottom two panels).

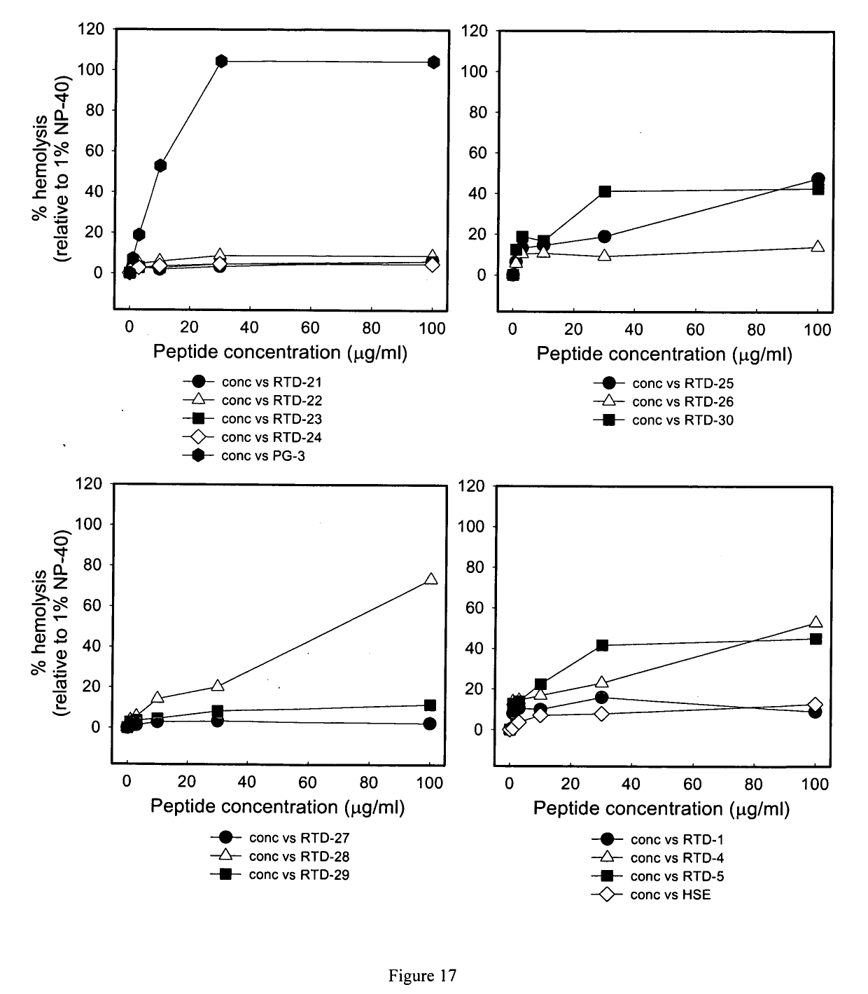

The cytotoxicity of selected theta defensins was also tested in additional cell lines, L929 cells and Hela cells. The cytotoxicity of theta defensins was compared to protegrin PG-10H (see structures in FIG. 1). Percent cell viability was tested after an 18 hour incubation of L929 or Hela 929 cells with various peptides at the indicated concentrations. The results are shown in FIG. 16. As was seen with HS68 fibroblasts, theta defensins showed low cytotoxicity, even at higher concentrations of peptide.

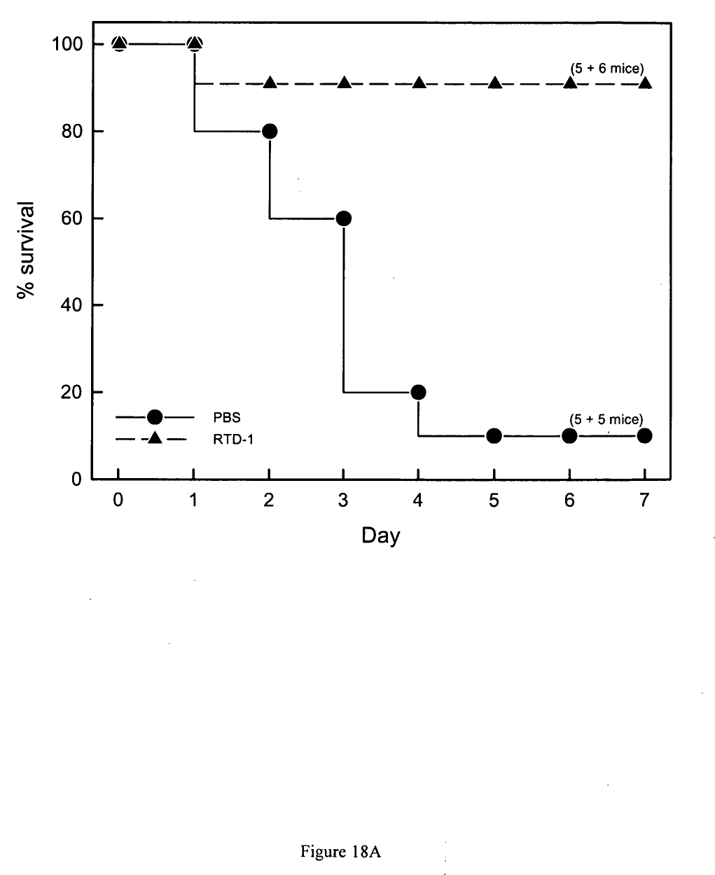

Cytotoxicity was also assessed using a hemolysis assay of RBCs. Hemolytic activity of each peptide was evaluated by testing for the percent of hemoglobin released from washed human erythrocytes following incubation with 0-8 μg/ml of each peptide. Peptides in 0.01% HOAc were diluted in phosphate buffered saline (PBS) and RBCs were diluted to 2% volume in 100 μl final reaction volumes. Cell suspensions were incubated 1 hour at 37° C. The cell suspension was centrifuged for 10 min at 22° C. Supernatants were removed, and absorbance was measured at 405 nm. Hemolytic activity of each peptide was calculated relative to 100% hemolysis by 1% NP-40 (see FIG. 17).

This example demonstrates that theta defensins have low cytotoxic activity.

### EXAMPLE VI

### Efficacy of Theta Defensins as Therapeutic Agents in an Experimental Polymicrobial Peritonitis Model

This example describes the efficacy of theta defensins in treating an experimental model of polymicrobial peritonitis.

Cecal ligation and puncture (CLP) was performed on adult Balb/C mice at time 0. Theta defensin peptides (5 mg/kg in 0.12 to 0.15 ml of PBS) were injected intravenously (tail vein) 4 h post-CLP. Control animal received 0.12 to 0.15 ml of PBS vehicle. Survival was followed for up to 3 weeks. Surviving peptide-treated animals were clinically normal for the entire period of observation.

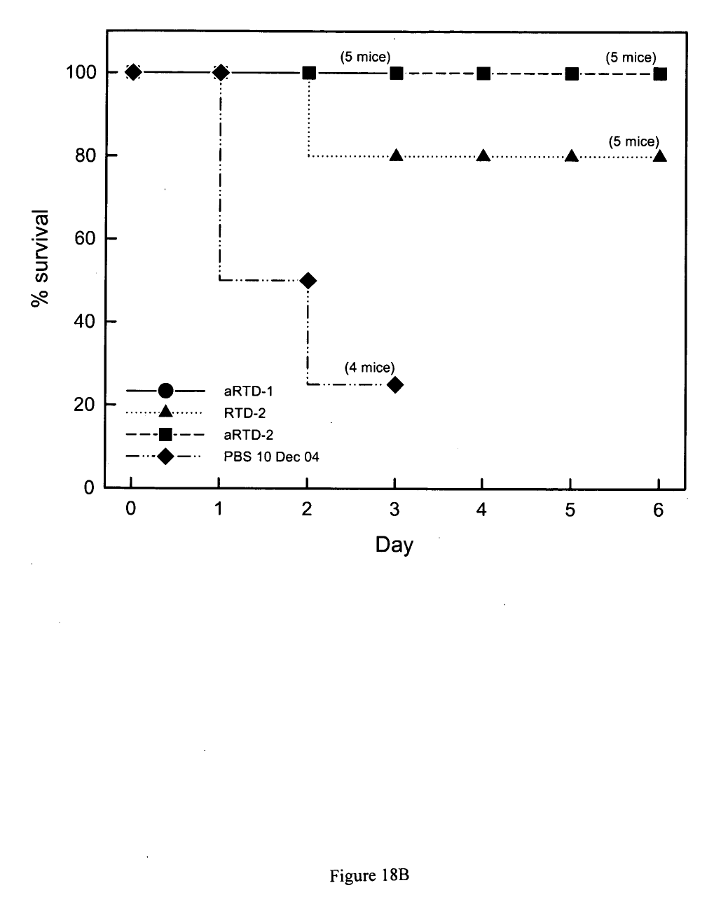

As shown in FIG. 18, administration of theta defensin 4 hours after perforation and when infection and inflammation are raging in the peritoneum was able to dramatically increase survival to nearly 100% versus very poor survival with no treatment. These results demonstrate that theta defensins are exceptionally effective at improving survival in a model of polymicrobial peritonitis.

### EXAMPLE VII

### Effect of Theta Defensins on Cytokine Release from Peripheral Blood Mononuclear Cells

This example describes the effect of theta defenins on cytokine release from peripheral blood mononuclear cells (PBMCs).

Human PBMCs were prepared by the dextran sedimentation method and resuspended in Iscove's Modified Dulbecco's Medium containing 10% fetal bovine serum. FIG. 19 shows TNFα release by human PBMCs under various conditions. Under conditions shown in A-C, aliquots containing 0.5−1×10⁶ PBMCs were incubated with 10 μg/ml lipopolysaccharide (LPS) for 2 hours at 37° C., 5% CO₂, then treated with RTD-1 (A: 10 μg/ml, B: 1 μg/ml, C, 0.1 μg/ml). Under conditions shown in D-E, a mixture of LPS (10 μg/ml) and RTD-1 (D: 10 μg/ml, E: 1 μg/ml, F: 0.1 μg/ml) were incubated for 2 hours at 37° C. then added to PBMC. Under conditions shown in G-L, control incubations were performed in which PBMCs were incubated with either 10 μg/ml LPS (G), 1 μg/ml phorbol 12-myristate 13-acetate (PMA) (H), varying concentrations of RTD-1 (1:10 μg/ml, J:1 μg/ml, K:0.1 μg/ml), or in media (L). Supernatants from these incubations were harvested after 3 days and soluble TNF was measured using Human TNF ELISA Kit II (BD Biosciences, San Diego). Results were obtained from duplicate incubations.

These results show that RTD-1 modulates extracellular release of TNF-a by human PBMC stimulated with LPS.

### EXAMPLE VIII

### Delivery of Theta Defensin Peptides Using Peptide Transduction

This example describes using peptide transduction to target theta defensins to cells.

One peptide transduction approach involves Tat-derived fusions. Studies performed over the last decade have identified peptides that are capable of spontaneously penetrating many cell types while displaying little or no toxicity, the prototype of these being an 11-residue peptide sequence (hereafter termed TAT) from the HIV Tat protein (Frankel and Pabo, Cell 55:1189-1193 (1988); Hallbrink et al., Biochim. Biophys. Acta 1515:101-109 (2001); Wadia and Dowdy, Curr. Opin. Biotechnol. 13:52-56 (2002). Remarkably, the mechanism that mediates this uptake is still unknown (Lindsay, Curr. Opin. Pharmacol. 2:587-594 (2002). Many studies have demonstrated that peptides, proteins, peptide-nucleic acids, liposomes, and small molecules may be efficiently targeted to various cell compartments as covalent fusions with TAT or similar sequences (Zhao and Weissleder, Med. Res. Rev. 24:1-12 (2004). Schwarze et al. demonstrated that intraperitoneal injection of a TAT-β-galactosidase fusion resulted in delivery of enzymatically active β-galactosidase to all tissues of the injected mouse (Schwarze et al., Science 285:1569-1572 (1999). Thus, protein transduction has proven to be a powerful tool for targeting proteins and other TAT-conjugates to cells in vivo (Wadia and Dowdy, supra, 2002).

Synthetic analogs of selected antimicrobial peptides are produced, for example, by extending the N-terminus of the antimicrobial peptide with the TAT-sequence (YGRKKRRQRRR) or the Arg9 sequence (RRRRRRRRR; shown to be even more efficient than TAT (Wender et al., Proc. Natl. Acad. Sci. USA 97:13003-13008 (2000)). The synthesis per se is a simple extension of the chain. However, for peptides that are cyclized, the sequence can be slightly modified by replacing an arginine, for example, at position eight in RTD-1 (see FIG. 1) with a lysine. This allows for a segment coupling of TAT or Arg9 to the antimicrobial peptide through the ε-amino group of the lysine in manner similar to that described previously (Cole et al., Proc. Natl. Acad. Sci. USA 99:1813-1818 (2002)). Briefly, Arg9 in RTD-1 is replaced with a Lys(carboxybenzyl) during synthesis. After peptide cyclization, the Cbz group is cleaved using HBr/AcOH. The TAT or Arg9 peptides are synthesized on chlorotrityl resin using Fmoc chemistry, and the fully protected peptide is cleaved with 1% trifluoroacetic acid (TFA) in dichloromethane. The peptide is then coupled through the ε-amino group of the lysine of RTD-1 using Opfp activation in dimethylformamide. Final deprotection is carried out in TFA. The fusion peptides are purified and characterized, and their anti-chlamydial and other antimicrobial activities and cytotoxicity profiles are determined in vitro. Uptake in vitro and in vivo are evaluated using specific anti-peptide antibodies.

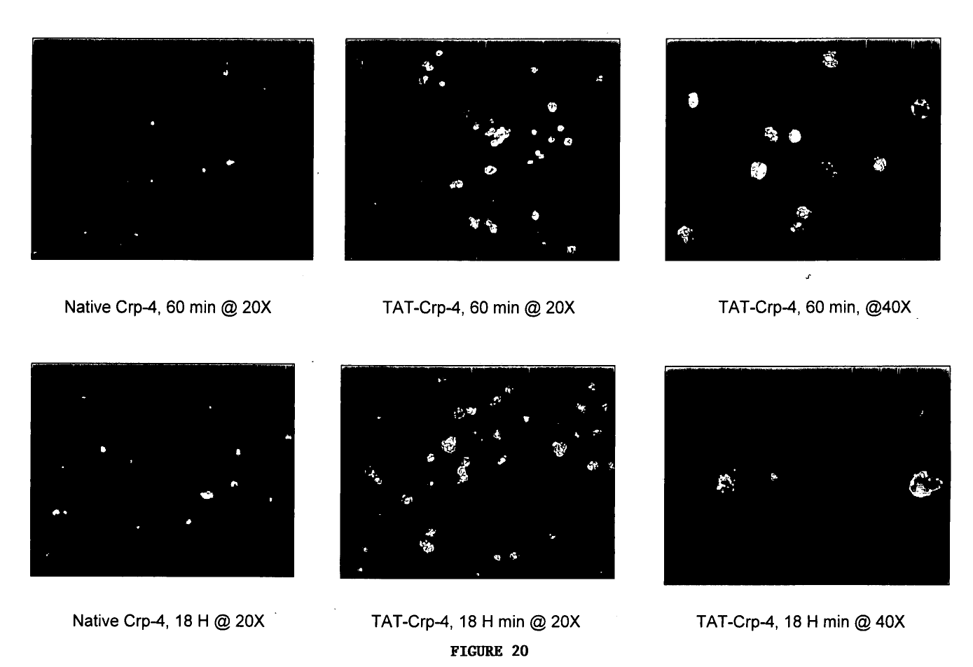

Exemplary targeting of a defensin peptide using TAT transduction is shown in FIG. 20. FIG. 20 shows transduction of TAT-Cryptdin 4 into 293T cells and visualization of TAT-Cryp4. Cryptdin 4 is an alpha-defensin having the sequence GLLCYCRKGHCKRGERVRGTCGIRFLYCCPRR. The cysteine connectivities are C1-6 (amino acids 4 and 29), 2-4 (amino acids 6 and 21) and 3-5 (amino acids 11 and 28). 293T cells were plated in a 6-well plate (3×10⁵/well) before the day of transduction in DMEM, 10% FCS, 1× Pen/Strep, overnight at 37° C. Medium was changed and TAT-Cryptdin 4 or cryptdin 4 was added to a final concentration of 40 μg/ml (1 ml/well). Peptide was incubated with cells for 60 min or 24 hr at 37° C. Cells were washed and trypsinized from the plate, washed 3× with PBS and then deposited on cytospin slides. Slides were fixed with 4% paraformaldehyde (PFA) for 10 min on ice, washed three times with PBS, permeabilized with 95% ethanol for 10 min on ice and washed three more times with PBS. Slides were incubated with anti-cryptdin-4 IgG for 60 min and washed. Slides were incubated with goat anti-rabbit IgG-FITC (fluoroscein isothiocyanate) and counterstained with propidium iodide.

An alternative approach for targeting theta defensin peptides is using a peptide carrier. The method is based on the findings of Morris et al. who reported that Pep-1 (KETWWETWWTEWSQPKKKRKV) efficiently shuttles peptide and proteins into a variety of cells in trans (Morris et al., Nat. Biotechnol. 19:1173-1176 (2001). Optimal Pep-1 mediated delivery of large and small peptides was achieved using a 10-20 molar excess of Pep-1, and no toxicity was observed. Moreover, appropriate targeting of imported proteins was observed. Pep-1 is synthesized as described previously be Morris et al., and Pep-1/antimicrobial peptide formulations are prepared in a manner similar to the approach described above.

Throughout this application various publications have been referenced. The disclosures of these publications in their entireties are hereby incorporated by reference in this application in order to more fully describe the state of the art to which this invention pertains. Although the invention has been described with reference to the examples provided above, it should be understood that various modifications can be made without departing from the spirit of the invention.

1. A method of inhibiting undesirable microbial growth in an individual comprising administering to said individual an effective amount of a theta defensin, whereby microbial growth is inhibited.

2. The method of claim 1, wherein said undesirable microbial growth is an infection.

3. The method of claim 1, wherein said undesirable microbial growth is polymicrobial.

4. The method of claim 1, wherein said undesirable microbial growth results in systematic inflammatory response syndrome, sepsis, septic shock, or immunoparalysis.

5. A method of inhibiting an effect of microbial growth in an individual comprising administering to said individual an effective amount of a theta defensin, whereby microbial growth is inhibited.

6. The method of claim 5, wherein said effect is an inflammatory response.

7. The method of claim 5, wherein said effect is an immunosuppressed state.

8. The method of claim 5, wherein said effect is an autoimmune disease.

9. The methods of claim 1, wherein said theta defensin is selected from a peptide selected from the peptides set forth in FIG. 1.

10. The method of claim 1, wherein said theta defensin is a cationic, arginine-rich cyclic peptide having each amino acid linked by a peptide bond and having one or more intrachain crosslinks, said intrachain crosslink formed between two amino acids, said theta defensin peptide or functional fragment lacks a free amino or carboxyl terminus, has less than 29 amino acids, and possesses antimicrobial activity.

11. The method of claim 1, wherein said theta defensin is a cationic, arginine-rich cyclic peptide having each amino acid linked by a peptide bond and having one or more intrachain crosslinks, said intrachain crosslink formed between two amino acids, said theta defensin peptide or functional fragment lacks a free amino or carboxyl terminus, has less than 29 amino acids, and improves the immune function of the individual.

12. The method of claim 1, wherein said theta defensin peptide has the amino acid sequence:
Xaa1-Xaa2-Xaa3-Xaa4-Xaa5-Xaa1-Xaa6-Xaa4-Xaa4-Xaa1-Xaa1-Xaa6-Xaa4-Xaa5-Xaa1-Xaa3-Xaa7-Xaa8,
wherein
Xaa1 independently is an aliphatic amino acid;
Xaa2 is an aromatic amino acid;
Xaa3 is Cys or Trp;
Xaa4 independently is Arg or Lys;
Xaa5 is Cys or Trp;
Xaa6 is Cys or Trp;
Xaa7 is Thr or Ser; and
Xaa8 is Arg or Lys.

13. The method of claim 1, wherein said theta defensin peptide has the amino acid sequence:
Xaa1-Xaa2-Xaa3-Xaa4-Xaa5-Xaa1-Xaa6-Xaa4-Xaa4-Xaa1-Xaa1-Xaa6-Xaa4-Xaa5-Xaa1-Xaa3-Xaa7-Xaa8,
wherein
Xaa1 independently is Gly, Ile, Leu, Val or Ala;
Xaa2 is Phe, Trp or Tyr;
Xaa3 is Cys or Trp;
Xaa4 independently is Arg or Lys;
Xaa5 is Cys or Trp;
Xaa6 is Cys or Trp;
Xaa7 is Thr or Ser; and
Xaa8 is Arg or Lys.

14. The method of claim 13, wherein said theta defensin has the amino acid sequence:
Gly-Phe-Cys-Arg-Cys-Leu-Cys-Arg-Arg-Gly-Val-Cys-Arg-Cys-Ile-Cys-Thr-Arg (SEQ ID NO:).

15. The method of claim 13, wherein Xaa1 is linked through a peptide bond to Xaa8.

16. The method of claim 13, wherein an intrachain crosslink is formed between two amino acids selected from the group consisting of:
Xaa3 at position 3 and Xaa3 at position 16;
Xaa5 at position 5 and Xaa5 at position 14; and
Xaa6 at position 7 and Xaa6 at positionl2.

17. The method of claim 16 wherein Xaa1 is linked through a peptide bond to Xaa8.

18. The method of claim 16, wherein said intrachain crosslink is a disulfide crosslink.

19. The method of claim 16, wherein said intrachain crosslink is a di-tryptophan crosslink.

20. (canceled)

21. The method of claim 17, having the amino acid sequence:
Gly-Phe-Cys-Arg-Cys-Leu-Cys-Arg-Arg-Gly-Val-Cys-Arg-Cys-Ile-Cys-Thr-Arg (SEQ ID NO:).

22-60. (canceled)


SEQUENCE LISTING (43 sequences)

SEQ ID NO: 1 - protein, 18 aa, Artificial Sequence
  synthetic construct
GFCRCLCRRGVCRCICTR

SEQ ID NO: 2 - protein, 18 aa, Macaca mulatta
GFCRCLCRRGVCRCICTR

SEQ ID NO: 3 - protein, 18 aa, Artificial Sequence
  synthetic construct
GFCRCLCRRGVCRCICTR

SEQ ID NO: 4 - protein, 18 aa, Macaca mulatta
GFCRCICTRGFCRCICTR

SEQ ID NO: 5 - protein, 18 aa, Artificial Sequence
  synthetic construct
GVCRCLCRRGVCRCLCRR

SEQ ID NO: 6 - protein, 18 aa, Macaca mulatta
GVCRCLCRRGVCRCLCRR

SEQ ID NO: 7 - protein, 18 aa, Artificial Sequence
  synthetic construct
GFCRCICTRGFCRCICTR

SEQ ID NO: 8 - protein, 18 aa, Artificial Sequence
  synthetic construct
GFCRCICTRGFCRCICTR

SEQ ID NO: 9 - protein, 18 aa, Artificial Sequence
  synthetic construct
RGVCRCLCRRGVCRCLCR

SEQ ID NO: 10 - protein, 18 aa, Artificial Sequence
  synthetic construct
RGVARCLCRRGVCRCLAR

SEQ ID NO: 11 - protein, 18 aa, Artificial Sequence
  synthetic construct
RGGRLCYCRRRFCVCVGR

SEQ ID NO: 12 - protein, 18 aa, Artificial Sequence
  synthetic construct
RGGRLCYCRRRFCVCVGR

SEQ ID NO: 13 - protein, 18 aa, Artificial Sequence
  synthetic construct
GGRLCYCRRRFCVCVGRR

SEQ ID NO: 14 - protein, 18 aa, Artificial Sequence
  synthetic construct
GGRLCYCRRRFCVCVGRR

SEQ ID NO: 15 - protein, 18 aa, Artificial Sequence
  synthetic construct
GGRLCYCRRRFCVCVGRR

SEQ ID NO: 16 - protein, 18 aa, Artificial Sequence
  synthetic construct
GGCRCYCRRRFCVCVCRR

SEQ ID NO: 17 - protein, 18 aa, Artificial Sequence
  synthetic construct
GGCRCYCRRRFCVCVCRR

SEQ ID NO: 18 - protein, 18 aa, Artificial Sequence
  synthetic construct
GGCRCYCRRRFCVCVCRR

SEQ ID NO: 19 - protein, 18 aa, Artificial Sequence
  synthetic construct
RGGCRCYCRRRFCVCVCR

SEQ ID NO: 20 - protein, 18 aa, Artificial Sequence
  synthetic construct
RGGCRCYCRRRFCVCVCR

SEQ ID NO: 21 - protein, 18 aa, Artificial Sequence
  synthetic construct
GFCRALCRRGVCRAICTR

SEQ ID NO: 22 - protein, 18 aa, Artificial Sequence
  synthetic construct
GFARCLCRRGVCRCIATR

SEQ ID NO: 23 - protein, 18 aa, Artificial Sequence
  synthetic construct
GFARCLARRGVARCIATR

SEQ ID NO: 24 - protein, 18 aa, Artificial Sequence
  synthetic construct
GFARALARRGVARAIATR

SEQ ID NO: 25 - protein, 18 aa, Artificial Sequence
  synthetic construct
GFCRCRCRRGVCLCICTR

SEQ ID NO: 26 - protein, 18 aa, Artificial Sequence
  synthetic construct
GFCRCRCTRGFCICICTR

SEQ ID NO: 27 - protein, 14 aa, Artificial Sequence
  synthetic construct
GFCRCRRGVCRCTR

SEQ ID NO: 28 - protein, 14 aa, Artificial Sequence
  synthetic construct
GVCICRRRFCLCRR

SEQ ID NO: 29 - protein, 14 aa, Artificial Sequence
  synthetic construct
GVCLCIRGRCRCRR

SEQ ID NO: 30 - protein, 18 aa, Artificial Sequence
  synthetic construct
GVCTCICRRRFCGCLCRR

SEQ ID NO: 31 - protein, 18 aa, Artificial Sequence
  synthetic construct
GICRCICTRGFCRCICVL

SEQ ID NO: 32 - protein, 18 aa, Artificial Sequence
  synthetic construct
GICRCLCRRGVCRCICVL

SEQ ID NO: 33 - protein, 18 aa, Artificial Sequence
  synthetic construct
GICRCICVLGICRCICVL

SEQ ID NO: 34 - protein, 18 aa, Artificial Sequence
  synthetic construct
GFCRCLCRRGVCRCICTR

SEQ ID NO: 35 - protein, 18 aa, Artificial Sequence
  synthetic construct
RGGGLCYCRRRFCVCVGR

SEQ ID NO: 36 - protein, 9 aa, Artificial Sequence
  synthetic construct
RCICTRGFC

SEQ ID NO: 37 - protein, 9 aa, Artificial Sequence
  synthetic construct
RCLCRRGVC

SEQ ID NO: 38 - protein, 11 aa, Artificial Sequence
  synthetic construct
YGRKKRRQRRR

SEQ ID NO: 39 - protein, 9 aa, Artificial Sequence
  synthetic construct
RRRRRRRRR

SEQ ID NO: 40 - protein, 32 aa, Artificial Sequence
  synthetic construct
GLLCYCRKGHCKRGERVRGTCGIRFLYCCPRR

SEQ ID NO: 41 - protein, 21 aa, Artificial Sequence
  synthetic construct
KETWWETWWTEWSQPKKKRKV

SEQ ID NO: 42 - protein, 9 aa, Artificial Sequence
  synthetic construct
GVCRCLCRR

SEQ ID NO: 43 - protein, 9 aa, Artificial Sequence
  synthetic construct
GICRCICVL